(19) United States
(12) Patent Application Publication
(10) Pub. No.: US 2015/0246132 A1
(43) Pub. Date: Sep. 3, 2015

(54) NON-COVALENT, SELF-ORGANZING HYDROGEL MATRIX FOR BIOTECHNOLOGICAL APPLICATIONS

(71) Applicants: TECHNISCHE UNIVERSITÄT DRESDEN, Dresden (DE); LEIBNIZ-INSTITUT FÜR POLYMERFORSCHUNG DRESDEN E.V., Dresden (DE)

(72) Inventors: Robert Wieduwild, Dresden (DE); Yixin Zhang, Dresden (DE); Carsten Werner, Dresden (DE); Mikhail Tsurkan, Dresden (DE); Uwe Freudenberg, Dresden (DE)

(73) Assignees: Technische Universität Dresden, 01069 Dresden (DE); Leibniz-Institut für Polymerforschung Dresden e. V., 01069 Dresden (DE)

(21) Appl. No.: 14/427,870

(22) PCT Filed: Sep. 13, 2013
(86) PCT No.: PCT/DE2013/100327

§ 371 Date: Mar. 12, 2015

(30) Foreign Application Priority Data
Sep. 13, 2012 (DE) 10 2012 108 560.9

(51) Int. Cl. A61K 47/42; A61K 35/33; A61K 9/48; A61K 31/727; A61K 47/10

(52) U.S. Cl. CPC A61K 47/42; A61K 31/727; A61K 47/10; A61K 9/4866; A61K 35/33

(57) ABSTRACT

The invention relates to the non-covalent, self-organizing hydrogel matrix for biotechnological applications containing a covalent polymer peptide conjugate, wherein the covalent polymer peptide conjugate includes conjugates of two or more peptides that are coupled to a polymer chain and the peptide sequence contains a recurring dipeptide motif (BA)ₙ wherein B is an amino acid having positively charged side chain, A is alanine and n is an integer between 4 and 20.

The invention refers to a non-covalent self-organizing hydrogel matrix for biological applications. Furthermore, the invention refers to hydrogel matrix spheres formed from the hydrogel matrix as well as a composite of the hydrogel matrix or the hydrogel matrix spheres with cells imbedded therein. Other aspects of the invention refer to a capsule for the targeted release of therapeutic reagents, a composition from the hydrogel matrix, chemicals and therapeutic reagents as well as a hybrid system from a non-spherical shaped hydrogel matrix and from hydrogel matrix spheres.

The design and the synthesis of self-organizing macromolecule systems for application in the area of “life science” and other areas is of great interest for chemistry, material sciences and biomedicine. Various hydrogels have awakened great expectations in their applicability in the biomedical area, such as for example in the area of active substance transport and tissue culture. A series of polymer matrices that are derived from living sources, such as for example matrigel and collagen hydrogels have shown to be superior relative to their high biocompatibility in the cell culture as compared to the known synthetic polymers. But such biomaterials from living sources have no definitive chemical composition thus preventing their broad application in biomedicine.

On the other hand, a system of polymeric matrices having a long shelf life can lead to a great variety of “made-to-measure” structures, that are suitable for a variety of applications. In this manner, many properties of the system can be influenced. An example at this point are different physical and biochemical properties for the cultivation of different cell types or different gelling times and degradation speeds for an implantation at various diseased locations. Finally, an ideal synthetic hydrogel system should not only be capable to imitate the biological function of various extracellular matrices (ECM) but also should offer the possibility to control these functions and to optimize them. The design of such adjustable materials for biomedical applications represents a big challenge. In particular this applies to biological investigations about the composition and the functions of the various extracellular matrices (ECM) for polymer- and material scientific reconstruction respectively for construction of such systems as well as chemical investigations about the control of processes.

Synthetic polymers such as polyethyleneglycol (PEG), polyvinylalcohol, poly(N-isopropylacrylamide), poly(lacticacid-co-glycolic acid) (PLGA) and copolymers of these and other polymers provide many useful systems for use in the biomedical area. On the one hand, polymers were developed as chemical structures having minimal interaction with biological systems. For example, the immune system oftentimes does not detect them as antigens, whereby complications of immunogenicity can be avoided. On the other hand, this advantage on the lack of function leads also to a lack of similarity of these polymers with important functions of the living system, primarily relative to the dynamic and the signaling in the extracellular matrix (ECM).

The conjugation of bio-macromolecules and synthetic polymers represents an interesting path in order to design the afore-stated hydrogel systems. The bio-macromolecules of choice can be either of synthetic origin, such as for example, peptides from the solid phase peptide synthesis and DNA from the solid phase-oligonucleotide-synthesis, or of biological origin with well defined chemical composition. Very important is that the bio-macromolecules exhibit no toxic properties and a low immunogenicity. Covalent as well as non-covalent methods can be used for crosslinking, whereby the non-covalent methods are of special research interest due to the possibility for the production of various gels. In addition, non-covalent, self organizing systems can realize embedding cells into a matrix-system, without relying on chemical reactions.

Incorporation of polysaccharide molecules in biohybrid material occurs more and more in order to achieve synthetic or semi synthetic materials. In particular, hyaluronic acid and heparin were used in a series of design concepts due to their biological activities and their biological availability. Heparin, a glycosaminoglycan (GAG) with the highest anionic charge density which occurs in a biopolymer is utilized due to its affinity for a multitude of important signal molecules. While heparin is a complex polymer which can be extracted from a biological source and respective samples differ from each other regarding mass distribution, composition of sugar monomers and the sulfating degree, dextransulfate and cyclodextrinsulfate are simpler oligosaccharides. In addition, some of these cyclodextrinsulfate compositions are obtained as pure chemical compounds.

The development of biocompatible hydrogel represents an interesting beginning for research in the area of material sciences and also in the area of biomedicine. Non-covalent self-organizing hydrogels or oligosaccharides containing hydrogels were developed within the last 10 years. In Kiicks et al. (N. Yamaguchi, B.-S. Chae, L. Zhang, K. L. Kiick, E. M/ Furst, Macromolecules 2005, 6, 1931-1940; N. Yamaguchi, K. L. Kiick, Journal of Controlled Release 2006, 114-130-142; K. L. Kiick, Soft Matter 2008, 4 29-37; F. J. Spinelli, K. L Kiick, E. M. Furst, Biomaterials 2008, 29, 1299-1306), the use of low molecular Heparin-Star-PEG-conjugate, that is, heparin (NMH) coupled to four-armed Star-PEG and the use of peptide-star-PEG-conjugate, that is a natural derivatives of peptides coupled to four-armed star-PEG, is described. After mixing of these two compounds, namely heparin-star-PEG and peptide-star-PEG, a hydrogel is formed in non-covalent way. The capacity of heparin with low molecular weight (LMWH) to bind multiple partners was exploited for the attachment or release of growth factors or other desired heparin-binding peptides, respectively proteins, at the non-covalent organized matrices. Thus, also the arrangement of these hydrogels with the dimer heparin-binding growth factors VEGF (VEGF=vascular endothelial growth factor) were utilized. An interesting result of the hydrogel networks that were mediated through a growth factor, is the ability for a respective receptor mediated gel-erosion. VEGF-networks, in presence of the VEGF receptors which control the proliferation and migration of vascular endothelial cell, can selectively compete and dissociate.

In the development and synthesis of hydrogels, increasingly bio-orthogonal reactions and photo-induced thiol-En reactions are utilized. The so-called click chemistry makes very selective and orthogonal reactions possible, which react with high efficiency under a series of mild conditions. Anseth et al. (S. B. Anderson, C.-C. Lin, D. V. Kuntzler, K. S. Anseth, Biomaterials 2011, 32, 3564-3574) have introduced a securely functioning synthesis strategy in which macromolecular precursors react by means of a copper-click-chemistry, which permits the direct encapsulation of cells within click-hydrogels. The mild chemical reaction between thiol and vinylsulfone was also intensively utilized for producing various hydrogels. Recently, this has led to a synergy of these chemical and biochemical reactions for the design and for the synthesis of a series of multi functionalized hydrogel systems.

In the inventors own work, a modular system of biohybrid hydrogels on the basis of covalent networked heparin and star-PEG was developed. (A. Zieries, S. Prokoph, P. Wenzel, M. Grimmer, K. Leventhal, W. Panyanuwat, U. Freudenberg, C. Werner, Journal of Materials Science: Materials in Medicine 2010, 21, 915-923; A. Zieris, S. Prokoph, K. R. Leventhal, P. B. Welzel, M. Grimmer, U. Freudenberg, C. Werner, Biomaterials 2010, 31, 7985-7994; U. Freudenberg, J.-U. Sommer, K. R. Leventhal, P. B. Welzel, A. Zieris, K. Chwalek, K. Schneider, S. Prokoph, M. Prewitz, R. Dockhorn, C. Werner, Advanced Functional Materials 2012, 22, 1391-1398; U. Freudenberg, A. Hermann, R B. Welzel, K. Stirl, S. C. Schwartz, M. Grimmer, A. Zieris, W. Panayanuwat, S. Zschoche, D. Meinhold, Biomaterials 2009, 30, 5449-5060; M. V. Tzurkan, K. R. Leventhal, U. Freudenberg, C. Werner, Chemical Communications 2010, 46, 1141; K. Chwalek, K. R. Leventhal, M. V. Tzurkan, A Ziereis, U. Freudenberg, C. Werner, Biomaterials 2011, 32, 9649-9657; M. V. Tzurkan, K. Chwalek, K. R. Leventhal, U. Freudenberg, C. Werner, Macromol Rapid Commun 2010, 31, 1529-1533) in which network properties can be gradually varied, while the content of heparin remains constant. As was shown, mesh width, swelling and elasticity modus correlate well with the degree of gel component networking. In addition, the secondary transformation of heparin within the biohybrid gels permits the covalent binding of cell adhesion promoting RGD-peptides. The biohybrid gels were utilized to demonstrate the effect of mechanical and biomolecular signals on the primary nerve cells and neuronal stem cells. The results show the cell specific interaction of synergistic signal giving and the potential of the biohybrid materials to selectively stimulate the cell destiny. Lately, the inventors own work combined the protease sensitive and insensitive cleaving locations for the extensive control about rates of degradation of star-PEG-heparin-hydrogel networks with orthogonally modulated elasticity, RGD-peptide presentation and VEGF-release. Enzymatic cleaving was massively accelerated when the protease access of the gels through non-enzymatic cleaving of ester bonds was increased. The effect of the degradation sensitivity of the gels was investigated for the three dimensional growth of human endothelial cells. Gels with accelerated degradation and a release of VEGF-release lead to a marked increase of the penetration of endothelial cells in vitro as also in the blood vessel density in chicken chorioallantois-membrane-test (HET-CAM) in vivo. Thus, the combination of protease sensitive and insensitive cleaving sites can reinforce the degradation of bio-responsive gel materials in such a way that increases the morphogenesis of the endothelial cells.

Artificial protein hydrogels which are synthesized by interaction of leucine-zipper domains have the ability to self-organize through the protein sequences. Tirrel et al. (W. Shen, K. Zhang, J. A. Komfiled, D. A. Tirrell, Nature materials 2006, 5, 153-15) have developed a hydrogel combined through the double helix domain. Investigations of the structural and dynamic properties of AC10A-hydrogels in closed systems showed that these multidomain protein chains have a strong tendency to form intramolecular loops. This leads to a rapid gel erosion. Thus, the system was improved, wherein it could be shown that the erosion speed of the protein hydrogel though exploitation of a selective molecular recognition, through a determined aggregation number and through orientation discrimination of twin helical domains, can be coordinated. Experiments have shown that the interaction between molecules during the self-organization and gelling function does not function as simple as a “key-in-lock” process. Instead, the dynamics and thermodynamics determines the entire system of physical and biochemical properties of the resulting polymer matrices. Since such physical and chemical parameters cannot be simply investigated and predicted, through the inventors' own work of the present invention, a screening method was applied in order to find an optimal self-organizing system of matrices through the synthesis of many different peptides and the investigation of their structure-function relationship.

The recognition between base pairs from two complementary DNA-sequences is likely the best characterized and most widely applied interaction between biomolecules. This base pair recognition is not only the topic of a multitude of genetic and biochemical research, but is also an increasingly useful tool in the material sciences. For example, the much promising DNA-origami technology was developed for the construction of nanostructures of any form and topology. Through DNA-self-organizing and/or enzyme catalyzed DNA ligation, DNA based hydrogel systems were recently developed. Luo et al. (S. H. Um, J. B. Lee, N. Park, S. Y. Kwon, C. C. Umbach, D. Luo, Nature materials 2006, 5, 797-801) have reported on the complete construction of a hydrogel from branched DNA. Since the DNA is an essential component in biology, these DNA-hydrogels are biocompatible, biologically degradable, can be efficiently produced and in simple manner they can be rendered into any desired form and size. Gelling processes of the DNA can be realized under physiological conditions. The coating of proteins and cells can be carried out in situ. In addition, the fine tuning of these hydrogels can be realized by adjusting the starting concentration and kinds of branched DNA monomers. The most important result was that the resultant polymatrices showed highly defined structures in the nanometer range and showed a good alignment with the prognosis regarding the DNA double helix structure.

Disadvantageously, the hydrogel system based on the peptide-Star-PEG conjugate and LMWH-Star-PEG-conjugate has proved to be very soft and thus not suitable for many construction processes. The peptide sequences of the AT-Ill-Peptide and the HIP-Peptide originate each from heparin binding protein antithrombin III (ATIII) and HIP (HIP=heparin/heparan sulfate interacting protein), each of which exhibit biological activities itself. In similar manner, the dimer growth factor respectively the VEGF-gel have the potential risk to produce an undesired reaction from the cells or from the host. Thus, the gel from the dimer growth factor and the growth factors themselves can be highly toxic because the growth factors are present in the body in only small amounts and also are effective at very low levels. An overdose is very dangerous and can lead from cancer to immediate death. Various protein-based hydrogels also carry the potential risk to elicit an immune response, since the artificial multi-domain-proteins are recognized through the host immune system as foreign antigens. The DNA-hydrogel for laboratory utilization can be produced at reasonable cost, while the synthesis at a larger scale can become very expensive. While it is chemically possible to incorporate other bioactive functional groups and/or chemical/physical reactive groups into the DNA-hydrogel, it would however raise the production cost considerably. Most chemical networking reactions for the polymerization would lead to a modification of the cell surface molecules and toxic for the cells. The thiol-En or thiol-maleimide-addition reactions are thus relatively mild, so that the alkene and the maleimide can react with free thiol groups of the cell surface molecules, while the thiol group in the polymer will have a disulfide-binding exchange reaction with the disulfide-bonds containing cell surface protein. Copper-free click chemistry represents the best suitable strategy for the chemical in situ gel formation. However, the cyclooctin structure is very lipophilic and could form a hydrophobic cluster in a polymer matrix. Moreover, the metabolism and the toxicity of the resulting triazole structure are unknown and have to be determined in clinical tests.

Object of the invention is to provided synthetic systems of polymer matrices by means of a rational design concept. With this system, an improvement of properties for biological and clinical applications is to be realized.

The solution of the object of the present invention consists in a non-covalent self-organizing hydrogel matrix for biotechnological applications comprising a covalent polymer-peptide conjugate, wherein the covalent polymer-peptide comprises two or more peptides which are coupled to a polymer chain. The peptide sequence includes a repeated dipeptide-motif (BA)ₙ where B is an amino acid with positively charged side chain, A is alanine and n is a number between 4 to 20 which represents the number of each repeating dipeptide module (BA) within the dipeptide-motif (BA)ₙ. The amino acid B is preferably arginine with a one-letter code R, or Lysine characterized by the one letter code K. The hydrogel according to the invention is suitable for the formation of a non-covalent hydrogel matrix. which based on the formation of the covalent polymer-peptide-conjugate exhibits polymer peptide-conjugate properties.

Thus, with the present invention an in situ self-forming hydrogel system is provided. The polymer matrices can be formed by simple mixing of two components that are completely compatible with cell-embedding experiments. In addition, a series of peptide-polymer conjugates were investigated in order to test their capacity to bond with an oligosaccharide to form a hydrogel. This approach does not only lead to a series of gel systems with various physical, chemical and biological properties, but also gives a view into the structure-function relationship. Thus, chemical, physical, biochemical and biological tests were carried out in relation to the resulting hydrogels. Since the peptide sequences are based on the simple (BA)ₙ motif, investigations on the structure-function relationship have shown that very simple changes in the sequences can lead to a multitude of gel property changes.

In accordance with the embodiment of the present invention, the polymer chain is formed by a linear multi-arm polyethyleneglycol (PEG). especially preferred is an embodiment where the polymer chain is formed of a four-armed polyethyleneglycol (Star-PEG). Amino acid B is preferably arginine or lysine. Besides L- and D-amino acids of arginine and lysine of the natural amino acids, but principally suitable are quasi all non-natural amino acids that are positively (basic) charged.

Corresponding to a specifically preferred embodiment of the present invention, the hydrogel matrix comprises in addition a highly negatively charged oligosaccharide. According to this embodiment, an oligosaccharide/peptide/polymer-system exists where the peptide is chemically conjugated to the polymer and the gel formation is carried out through mixing the peptide-polymer-conjugate and the oligosaccharide. The non-covalent macromolecular self-organization is also induced by the interaction of the peptide and the oligosaccharide. The choice of the polymer and the oligosaccharide can lead to various gel properties including the flow behavior, the gelling condition and the gelling speed as well as adjustable affinity of peptides interacting with bioactive proteins, for example, growth factors or morphogen. However, the greatest multitude in gel properties is surprisingly realized through changes of a very simple and repeating peptide sequence motif, wherein according to the concept of the present invention the corresponding hydrogel matrix is principally also possible without oligosaccharide. In this manner, the flexible design of the peptide sequence can lead to a broad variety of gel properties, that not only lead to the above-stated rheological properties, the gelling condition, the gelling speed and protein binding properties, but also leads to properties such as for example, the biological degradation due to proteolytic hydrolysis or other enzymatic activity such as light impact sensitivity.

The highly negatively charged oligosaccharide, according to an advantageous embodiment, is a sulfated or phosphorylated oligosaccharide, preferably selected from a group of oligosaccharides which comprises heparine, dextransulfate, α-cyclodextrinsulfate, β-cyclodextrinsulfate, γ-cyclodextrinsulfate, α-cyclodextrinphosphate, β-cyclodextrinphosphate, γ-cyclodextrinphosphate. In an especially preferred embodiment for an oligosaccharide/peptide/polymer system the hydrogel matrix comprises heparin as oligosaccharide, which originates from the mucosa of pig intestine or bovine lung tissue. Heparin is preferably of pharmaceutical quality. In an alternative embodiment the hydrogel matrix comprises dextransulfate as oligosaccharide, which preferably has a molecular weight in the range of 4 kDa to 600 kDa. Preferred is the use of dextransulfate of pharmaceutical quality. If the hydrogel matrix contains cyclodextrinsulfate, then it is preferably α-cyclodextrinsulfate, β-cyclodextrinsulfate, γ-cyclodextrinsulfate of pharmaceutical quality, wherein the sulphation degree of three sulfates per molecule up to a complete sulphation degree. if the hydrogel matrix contains α-cyclodextrin phosphate, β-cyclodextrinphosphate, γ-cyclodextrinphosphate then it is of pharmaceutical quality, wherein the degree of phophorylation of three phosphate groups per molecule can be up to the complete phosphorylation.

According to a further embodiment of the present invention the hydrogel matrix comprises a chemical group that is light-cleavable between the polymer chain and the peptide sequence which includes the repeated dipeptide-motif (BA)ₙ. The hydrogel matrix can also comprise the pH sensitive chemical linker between polymer chain and peptide sequence which includes the repeating dipeptide-motif (BA)ₙ. In accordance with another embodiment of the present invention the hydrogel matrix also comprises an enzymatic cleavable linker between the polymer chain, preferably a PEG molecule, and the peptide sequence which includes the repeating dipeptide-motif (BA)ₙ. The hydrogel comprises as enzymatic cleavable linker also an oligonucleotide sequence which is a nuclease-active substrate.

The modification of the peptide can lead to a further development of the hydrogel function through the insertion of different markers, for example fluorescence marking for monitoring the matrices in vivo and in vitro and for the further development of the active-compound-conjugation for an active compound release. Also, since the gel formation is induced by two chemically defined components, the matrix-system can be formed layer by layer, in order to place a peptide-polymer-Conjugate and/or an oligosaccharide with a certain function at predetermined layer with high precision. The layer by layer method, in combination with the above-stated embodiment of the light sensitive hydrogel matrix, renders possible a design and construction of sophisticated bioactive and biocompatible nanostructure and nano units.

All components in the hydrogel system according to the present invention can be produced and retained in a comparably inexpensive manner. Heparin, dextransulfate and cyclodextrinsulfate and also maleimide functionalized PEG-polymer are available at relatively low cost from commercial sellers. Peptides can be synthesized in a solid-phase-peptide-synthesizer in the lab on the scale of grams at relatively low cost. Advantageously, the hydrogel matrix in accordance with the above-described embodiments exhibits an elasticity module of at least 10 Pa.

The self-organizing system can be also used in a micro-fluid system in order to produce hydrogel balls as well as cells to be embedded into the hydrogel balls. A further aspect of the present invention thus refers to hydrogels with a self-organizing matrix forming hydrogel. Into a self-organizing hydrogel matrix formed from the hydrogel according to the present invention or into the hydrogel balls according to the present invention, as already noted, cells can be embedded via a corresponding method resulting in a corresponding composite. Hereby, the cells are preferably selected from a group which comprises mammalian cells, insect cells, bacteria cells and yeast cells. If the cells are mammalian cells, different cancer cell lines, fibroblast cells, pluripotent stem cells, induced pluripotent stem cells, human T-cells or human B-cells, can be advantageously selected. Cells embedded in a hydrogel matrix or respectively, hydrogel balls can be utilized for the production of proteins, wherein the protein preferably comprises therapeutic monoclonal antibodies.

A further aspect of the present invention refers to capsules for the targeted release of therapeutic reagents, wherein via a corresponding method therapeutic reagents are encapsulated with the above-described hydrogel matrix or the above-described hydrogel balls. The group of each of the utilized therapeutic reagents comprises preferably mammalian cells, insect cells, bacteria, yeast cells, anti-cancer compound, anti coagulation compounds, anti inflammatory compounds, immune-suppressive compounds, therapeutic antibodies, diagnostic reagents, hormones, growth factors, cytokine, small molecules as inhibitors for growth factors, small molecules as inhibitors for cytokines, aptamer-inhibitors for growth factors and aptamer-inhibitors for cytokine.

A further aspect of the invention refers to a composition of a non-covalent self-organizing hydrogel matrix in one of the above-described embodiments and chemicals and therapeutic reagents, wherein in the nascent therapeutic hydrogel a gradient of chemicals and reagents is produced. This means, it is possible through a suitable method to produce a gradient of chemicals and reagents in the therapeutic hydrogel matrices respectively the hydrogel balls according to the present invention. Possible therapeutic chemicals and reagents which form gradients in the hydrogel matrix are preferably anti-coagulation compounds, anti inflammatory compounds, immune-suppressive compounds, therapeutic antibodies, diagnostic reagents, hormones, growth factors, cytokine, small molecules as inhibitors of growth factors, small molecules as inhibitors for cytokine, aptamer-inhibitors for growth factors as well as aptamer-inhibitors for cytokine.

Finally, a further aspect of the present invention is a hybrid system of a hydrogel matrix according to the present invention, which, on the one hand are not in spherical shape, and hydrogel balls on the other hand. Hereby, the non-spherical hydrogel matrix and the hydrogel balls each exhibit a different chemical composition and one component of the hybrid system is controllable through light, through selective chemical degradation or through enzymatic digestion.

Other features and advantages of the present invention will be more readily apparent upon reading the following description of currently preferred exemplified embodiments of the invention with reference to the accompanying drawing, in which:

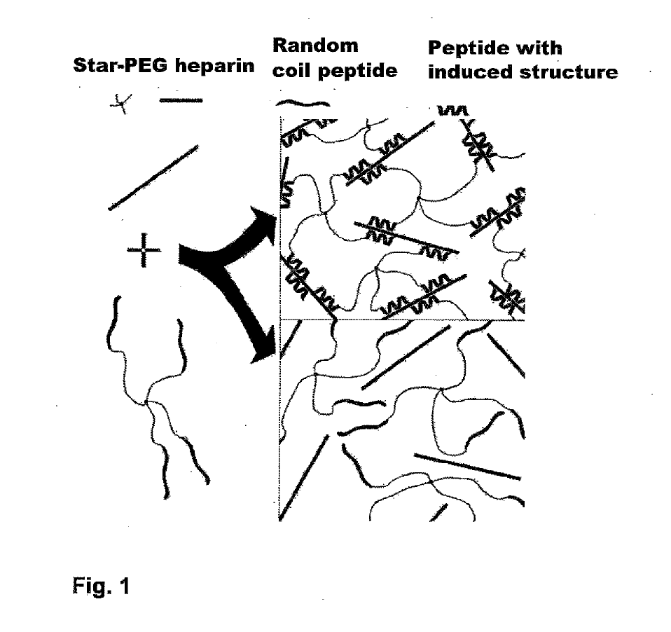

FIG. 1 is a schematic illustration of a selective method for detecting he formation of a hydrogel with heparin,

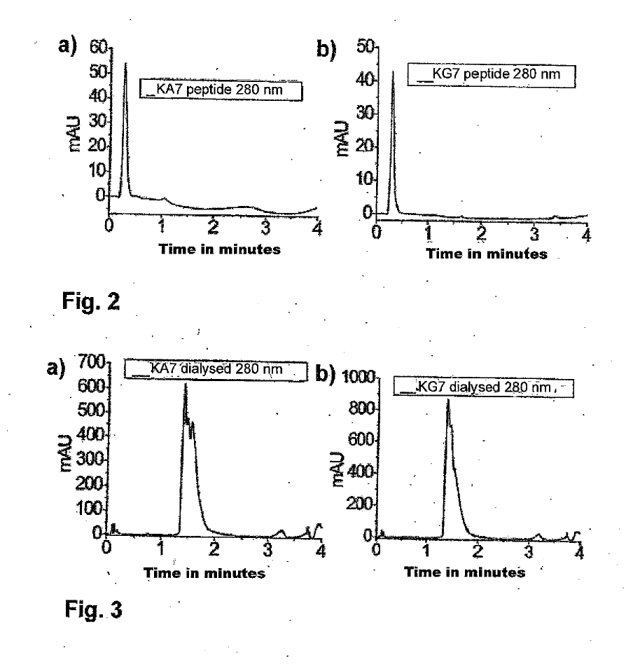

FIG. 2 the results of the reverse-phase-ultrahigh pressure fluid chromatography (UPLC) of purified peptides,

FIG. 3 the results of the reverse-phase-ultrahigh pressure fluid chromatography (UPLC) of purified peptide-four-arm polyethyleneglycol-conjugate (peptide-star-PEG),

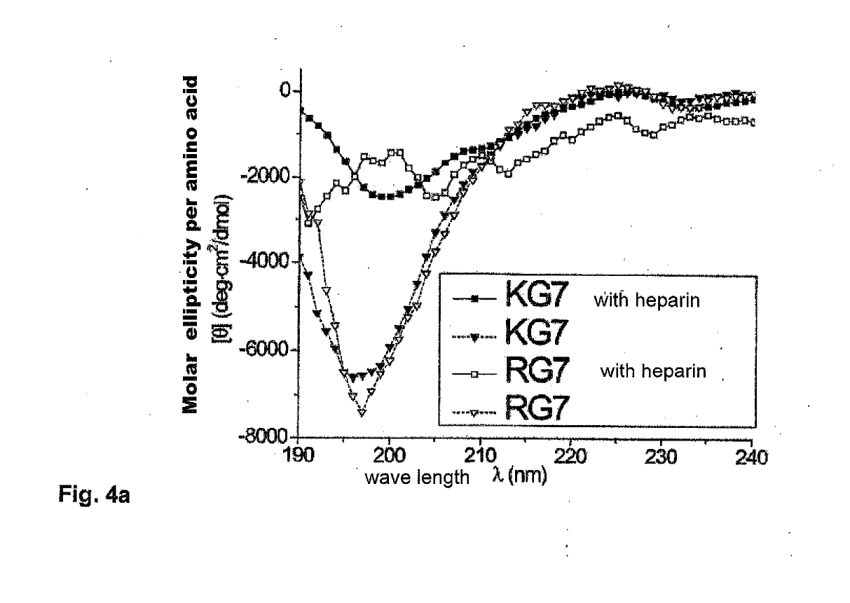

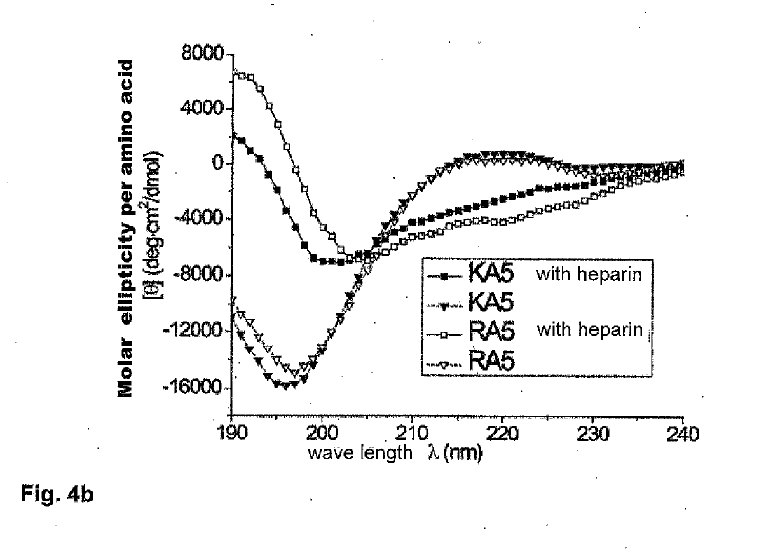

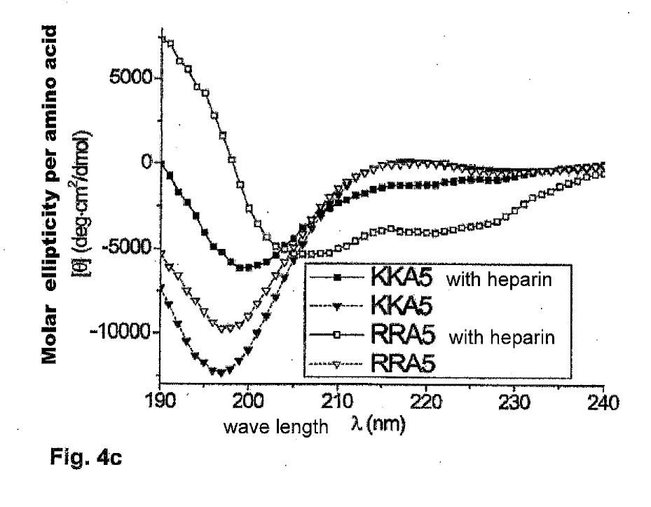

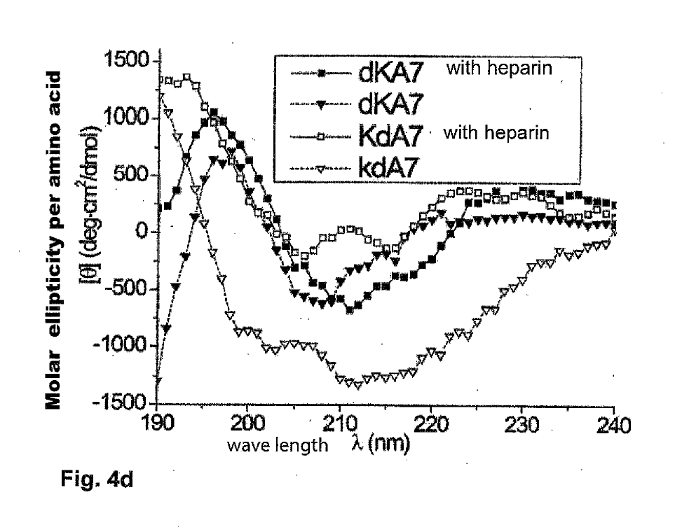

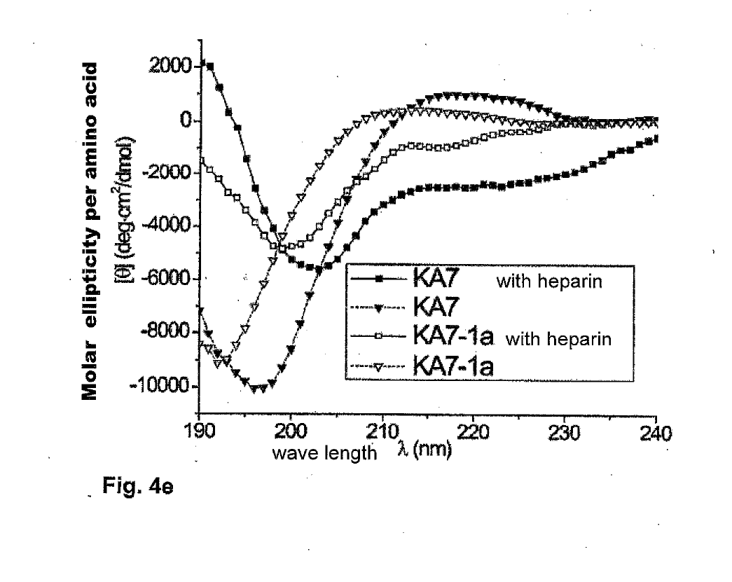

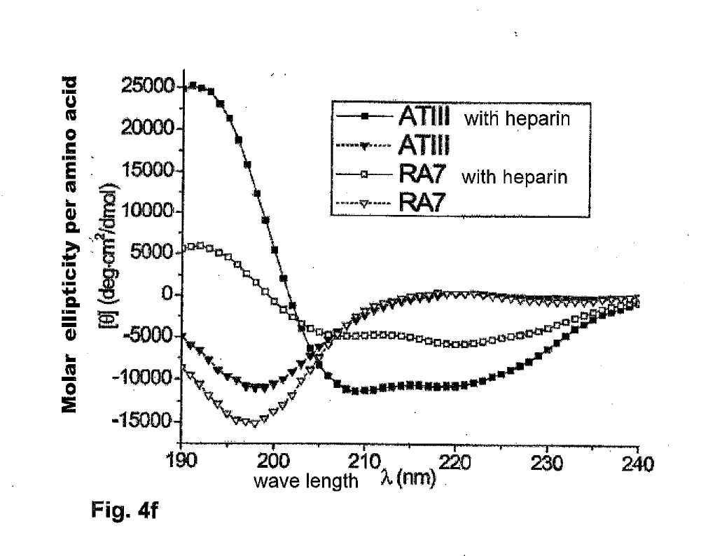

FIG. 4a-f an analysis of a heparin-dependent structural change through circular dichroism-spectroscopy,

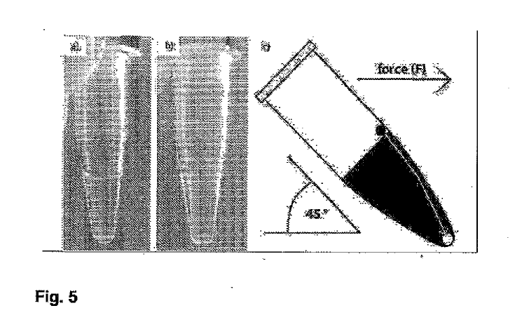

FIG. 5 a schematic illustration of a high throughput analysis of the mechanical properties of the hydrogels,

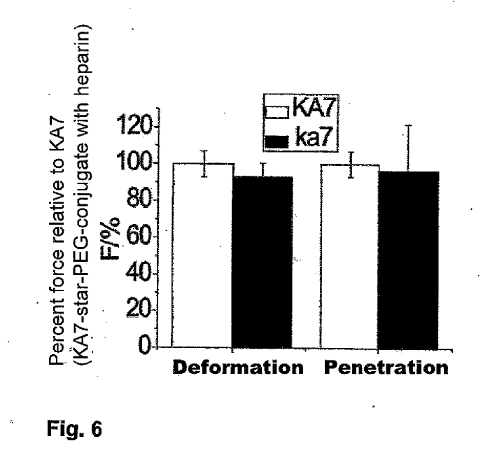

FIG. 6 the stability of a lysine and alanine-based hydrogel with comparison of L- and D-amino acids.

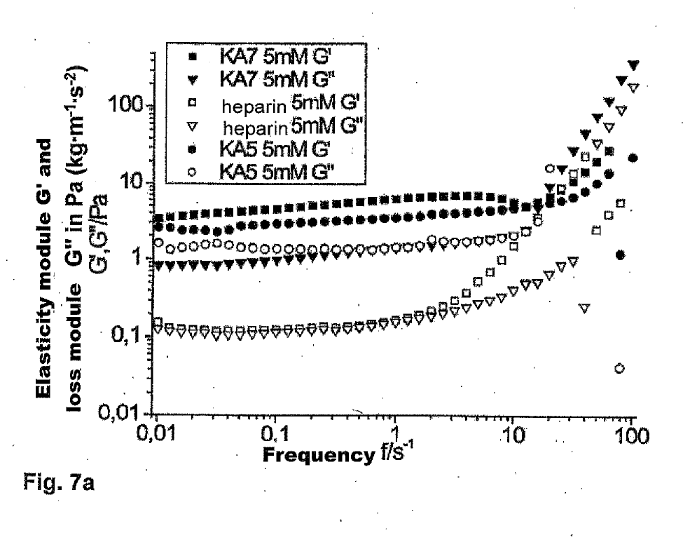

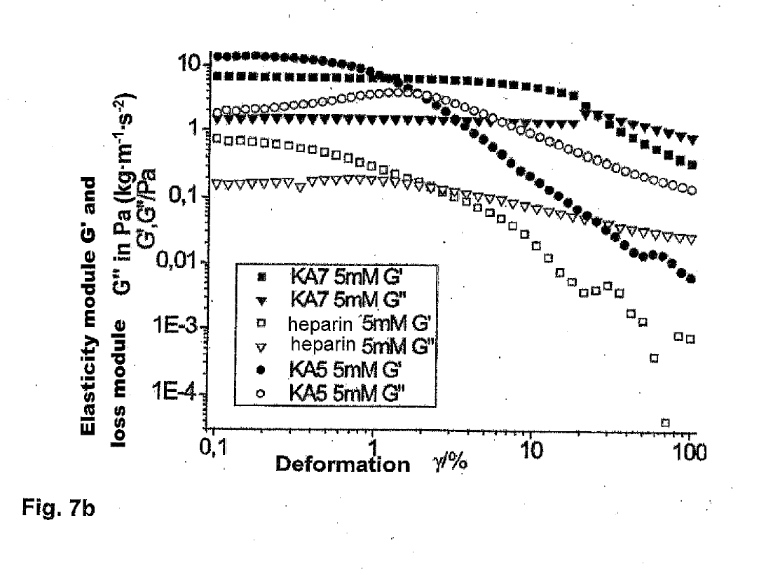

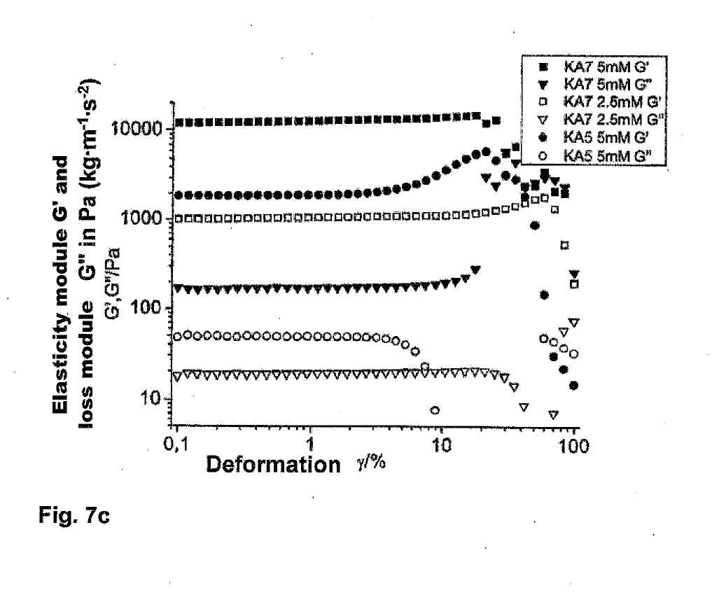

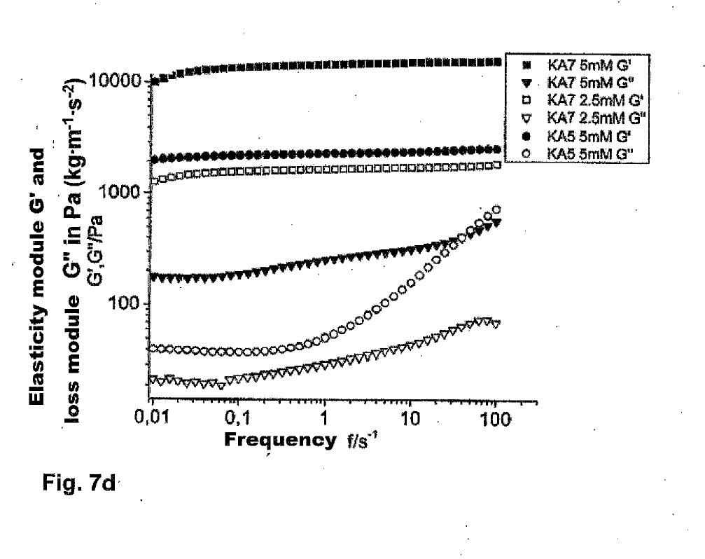

FIG. 7a-b the flow behavior of each single peptide-star PEG conjugate and 14-kDa-heparin,

FIG. 7c-d the flow behavior of peptide-star-PEG conjugate with 14-kDa heparin,

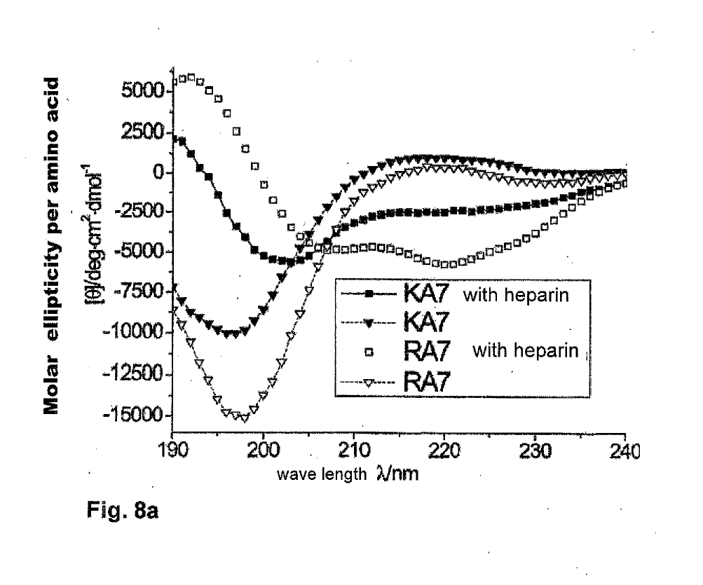

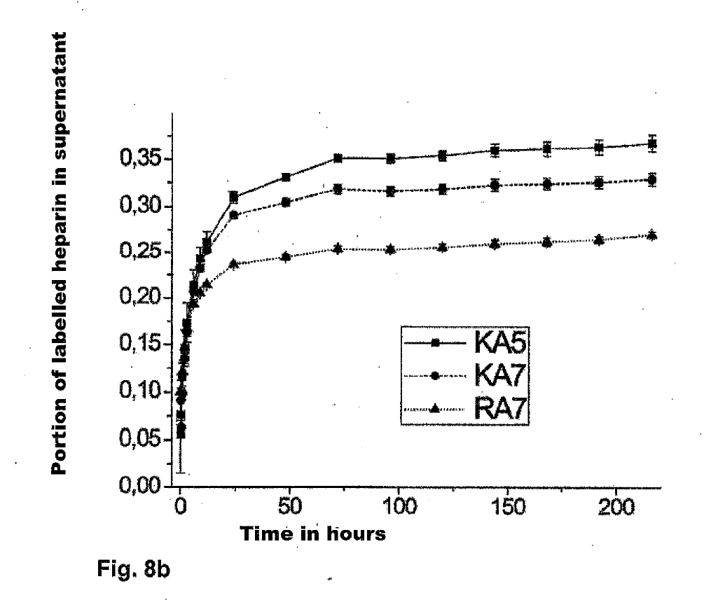

FIG. 8a analysis of a heparin dependent structural change through circular dichroism-spectroscopy,

FIG. 8b result of investigating the erosion of the hydrogel through mixing together of peptide-star-PEG conjugate with a TAMRA labeled 14-kDa heparin;

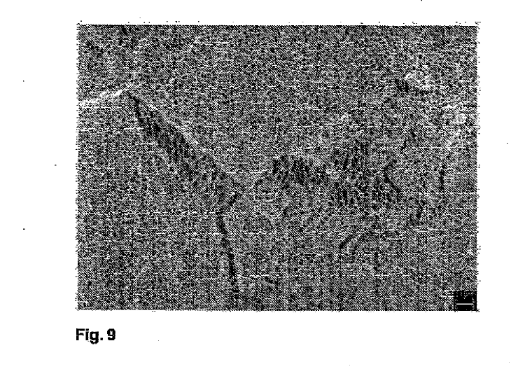

FIG. 9 a scanning microscopic image of KA7-star-PEG-hydrogel with heparin,

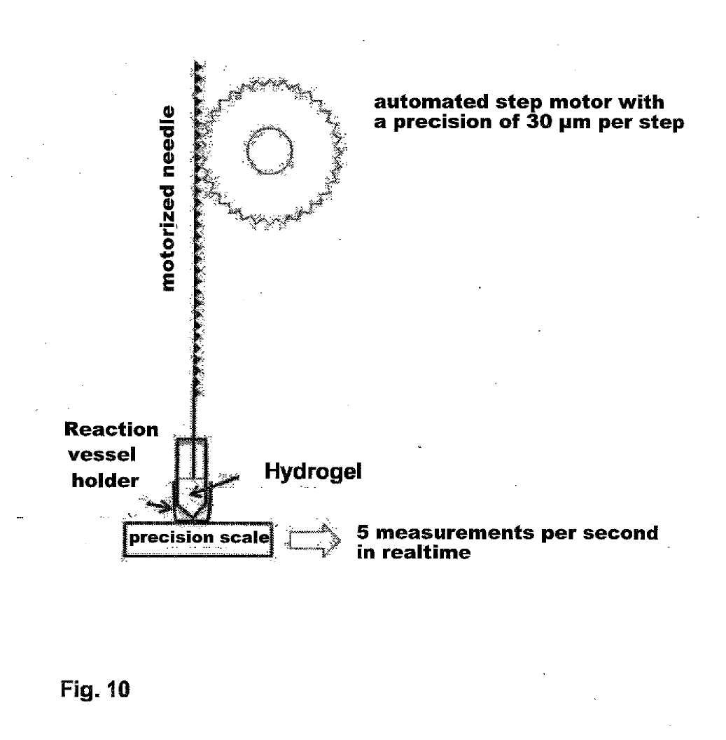

FIG. 10 a device for analysis of the gelling time of a hydrogel,

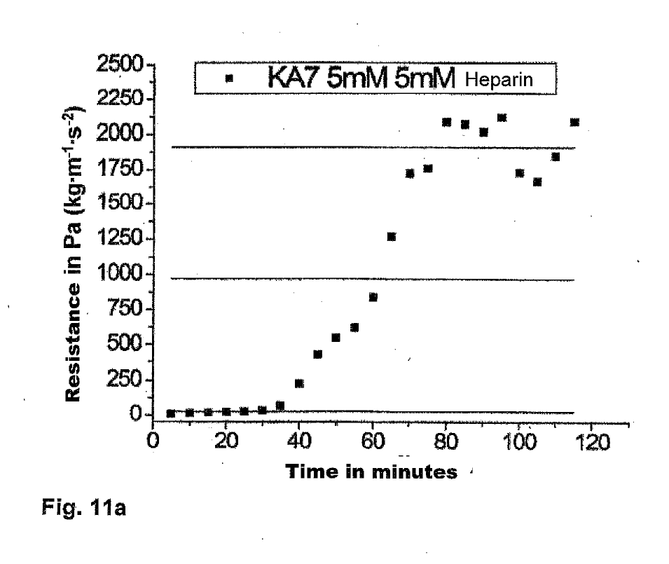

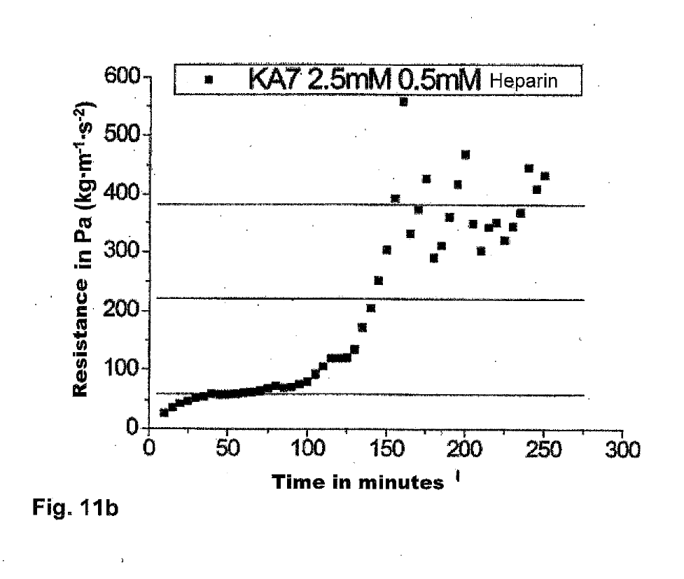

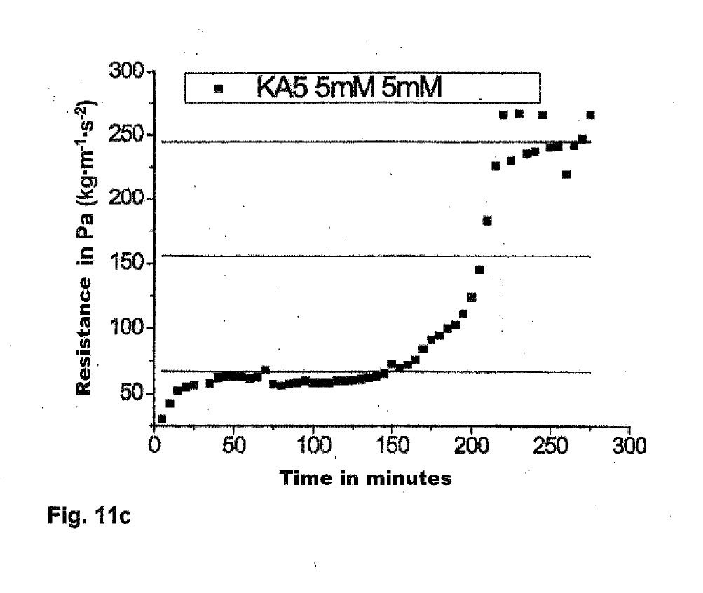

FIG. 11a-c the result of the analysis of the gelling time of a hydrogel,

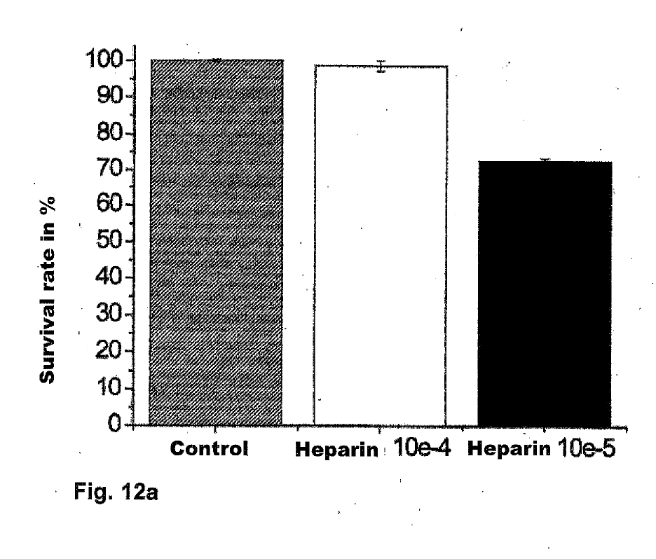

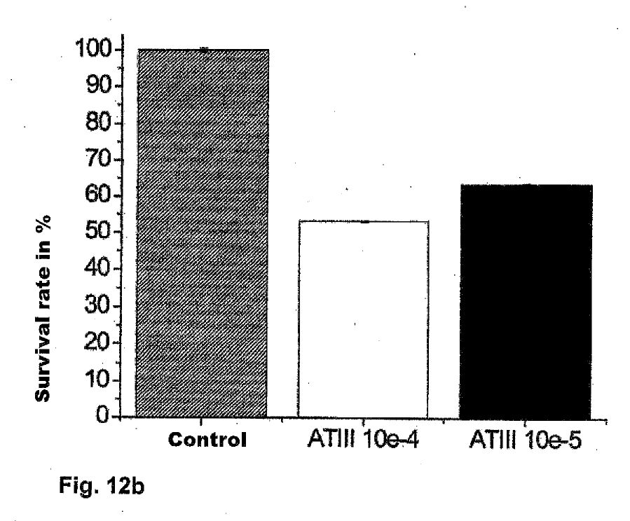

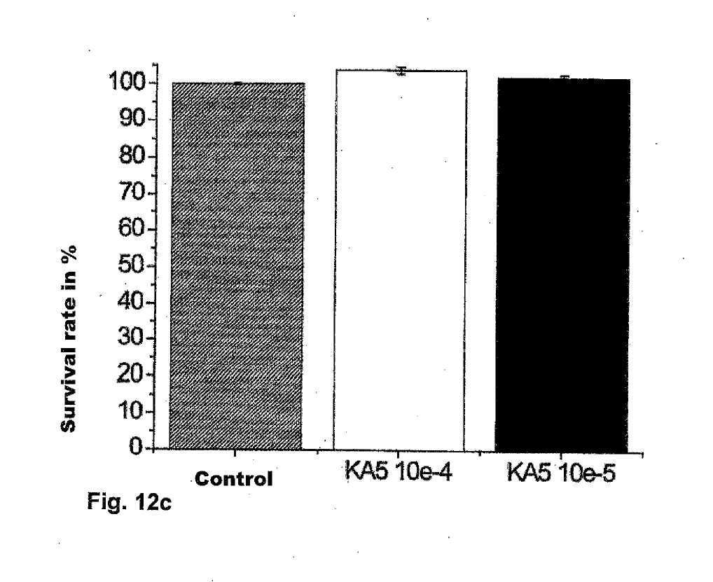

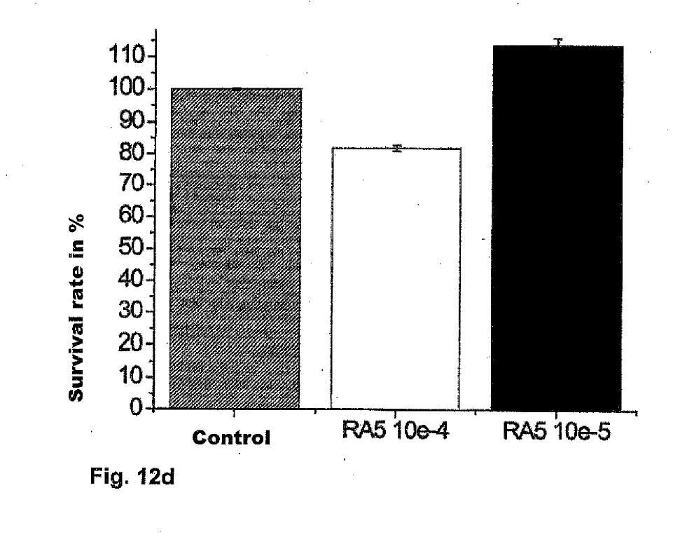

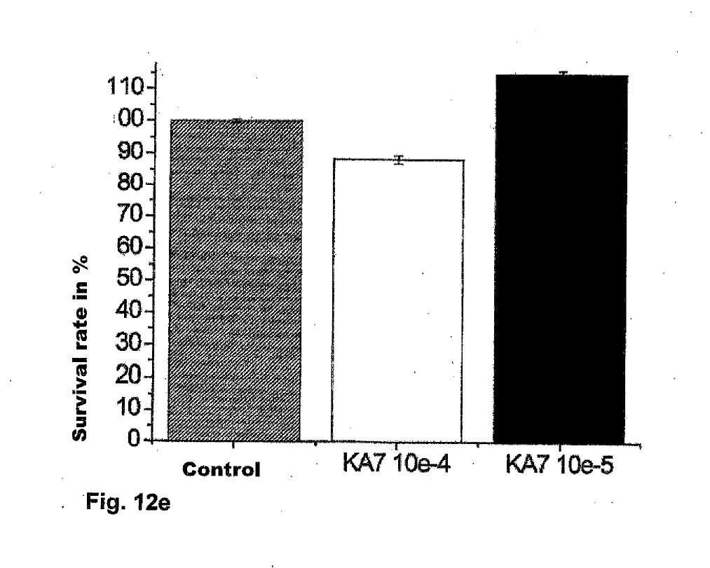

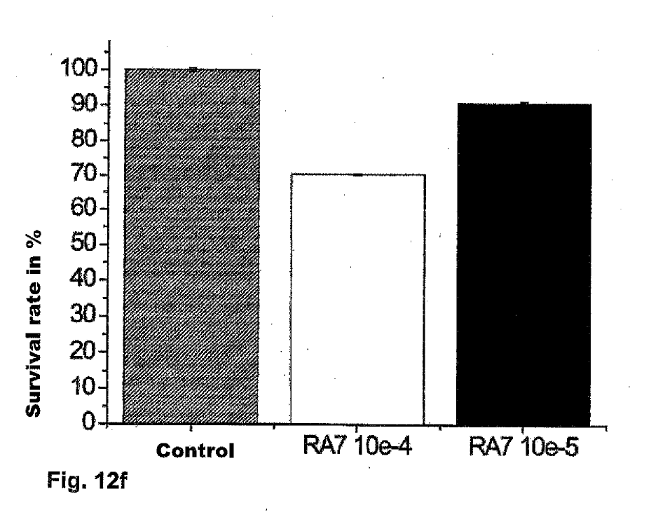

FIG. 12a-f a toxicity test for various peptide-star-PEG-conjugates and 14-kDa heparin and

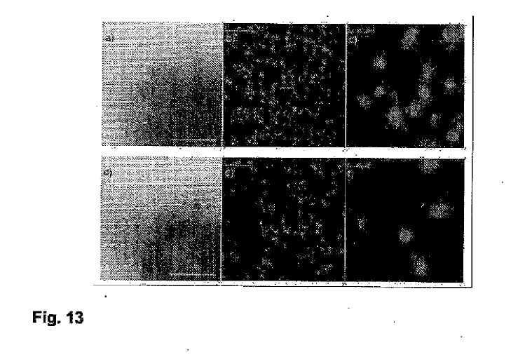

FIG. 13a-f the results of a viability test and the structure of embedded human fibroblasts (HDFn) from the skin of newborns with a hydrogel.

A simple self-repeating peptide motif which can be simply modified to lead to various binding properties at certain biomolecules is of great interest in biochemistry, biotechnology and in the biomaterial sciences. For example, such a system can be utilized to design adjustable self-organizing non-covalent matrix systems. Heparin was used as a starting compound in order to synthesize a covalent hydrogel platform to support cell replacement therapies. Following is a library of peptides, which are each conjugated to a four-armed polyethylene glycol (star-PEG) which serves as polymer chain in the examples of the embodiments. The library leads to the determination of a minimal heparin binding peptide motif (BA)ₙ wherein B is an amino acid residue, for example of arginine or lysine, and wherein A is alanine and n is a number between 4-20. The repetition of this motif or a single amino acid mutation leads to a multitude of physical and biochemical properties of the resulting heparin dependent self-organizing hydrogel.

FIG. 1 shows schematically a selection method for the detection that the specific peptide motif coupled to a four-armed polyethyleneglycol (star-PEG) can form a hydrogel with 14 kDa heparin. FIG. 1 makes clear that the hydrogel formation with a heparin induced structural change coincides with the (BA)ₙ-peptide motif.

Table 1 shows first the library of synthesized peptides. Shown are the sequences, the abbreviations and the molecular weight of the peptides.

TABLE 1

| | Sequence | | Molecular |
|---|---|---|---|
| | identifiers | Peptide | weight |
| Name | (SEQ ID NO) | sequence | [10⁻³ kg/mol] |
| ATIII | 1 | CWGGKAFAKLAARL | 2010,44 |
| | | YRKA | |
| KA1 | 2 | CWGGKA | 620,72 |
| KA3 | 3 | CWGGKAKAKA | 1019,22 |
| KA5 | 4 | CWGGKAKAKAKAKA | 1417,72 |
| KA7 | 5 | CWGGKAKAKAKAKA | 1816,22 |
| | | KAKA | |
| dKdA7 | 6 | cwGGkakakakaka | 1816,22 |
| | | kaka | |
| dKA7 | 7 | CWGGkAkAkAkAkA | 1816,22 |
| | | kAkA | |
| KdA7 | 8 | CWGGKaKaKaKaKa | 1816,22 |
| | | KaKa | |
| KA7-1a | 9 | CWGGKAKAKAKaKA | 1816,22 |
| | | KAKA | |
| KKA5 | 10 | CWGGKKAKKAKKAK | 2058,57 |
| | | KAKKA | |
| KG1 | 11 | CWGGKG | 606,69 |
| KG3 | 12 | CWGGKGKGKG | 977,13 |
| KG5 | 13 | CWGGKGKGKGKGKG | 1347,57 |
| KG7 | 14 | CWGGKGKGKGKGKG | 1718,01 |
| | | KGKG | |
| KKG5 | 15 | CWGKKGKKGKKGK | 1988,42 |
| | | KGKKG | |
| RA1 | 16 | CWGGRA | 648,74 |
| RA3 | 17 | CWGGRARARA | 1103,28 |
| RA5 | 18 | CWGGRARARARARA | 1557,82 |
| RA7 | 19 | CWGGRARARARARA | 2012,36 |
| | | RARA | |
| RRA5 | 20 | CWGRRARRARRARR | 2338,77 |
| | | ARRA | |
| RG1 | 21 | CWGGRG | 634,71 |
| RG3 | 22 | CWGGRGRGRG | 1061,19 |
| RG5 | 23 | CWGGRGRGRGRGRG | 1487,67 |
| RG7 | 24 | CWGGRGRGRGRGRG | 1914,15 |
| | | RGRG | |
| RRG5 | 25 | CWGRRGRRGRRGRR | 2268,62 |
| | | GRRG | |

Following are the one letter codes for the respective amino acids and (in parenthesis) opposite thereto their 3-letter codes:

A is the abbreviation for Alanine (Ala)

### C for Cysteine (Cys)

### F for Phenylalanine (Phe)

### G for Glycine (Gly)

### K for Lysine (Lys)

### L for Leucine (Leu)

### R for Arginine (Arg)

### W for Tryptophan (Trp) and

### Y for Tyrosine (Tyr)

L-amino acids are marked by the use of upper case letters, D-amino acids by lower case letters.

All peptides shown in Table 1 are produced by utilizing a standardized-fluorenylmethoxycarbonyl chemistry (FMOC chemistry) on a solid phase with 2-(1H-benzotriazol-1-yl)-1,1,3,3 tetramethyluronoiumhexafluorophosphate-activation (HBTU-activation) in an automatic solid phase peptide synthesizer (ResPep SL, Intavis, Cologne, Germany). To obtain good peptide quality, each amino acid was coupled two times with the fivefold excess, wherein all non-reacting amino groups were protected with acetic acid anhydride. For cleaving the peptide from the resin, the resin was treated for one and one half hour with a mixture of trifluoroacetic acid (TFA) triisopropylsilane(TIS)/water/dithiothreitol (DTT), wherein these components are present in a ratio of 90 (v/v):2.5 (v/v):2.5 (v/v):2.5 (m/v).

The peptides were dissolved in water, which contained 2 mg/ml tris(2-carboxyethyl)phosphine (TCEP). The peptide purification was carried out by means of reverse-phase high pressure liquid chromatography (UPLC) on a preparative HPLC-device (Prostar™, Agilent Technologies, Santa Clara, USA) which was provided with a preparative C18-column (AXIA™ 1001 A grain size 10 μm, 250×30 mM, Phenomenex Torrance USA). The peptide was eluted from the column by utilizing a gradient of 5% to 100% solvent B at 20 ml/min, wherein solvent A is 0.1% trifluoroacetic acid (TFA) in water and solvent B is 0.1% TFA and 5% water in acetonitril.

The purity was confirmed through analytical reverse-phase ultrahigh pressure liquid chromatography (UPLC Aquity™ with UV detector, Waters, Milford Mass., USA) provided with an analytical C18-column (AQUITY™ UPLC BEH C18, grain size 1.7 μm, 50×2.1 mM, Waters, Milford, Mass., USA) by utilizing an isocratic gradient and an electrospray-ionisation-mass-spectrometry (ESI-MS) (AQUITY™ TQ detector, Waters, Milford, Mass., USA). The peptide was dry frozen into a white powder (CHRIST ALPHA™ 2-4LD plus+ vacuubrand RZ6) and at 4° C. under dry conditions stored for not more than one week prior to further treatment.

FIG. 2 shows the results of the reverse phase ultrahigh pressure liquid chromatography (UPLC) of purified peptides at 280 nm by utilizing an analytical C18 column and an isocratic gradient. The sample peptides from the library shown in FIG. 2 are a) CWGGKAKAKAKAKAKAKA (KA7) and b) CWGGKGKGKGKGKGKGKG (KG7)
The synthesis of the peptide-star-PEG-conjugates for use in the hydrogel self-organization were carried out through Michael-addition-reactions between maleimide-terminal four-armed PEG and cysteine-terminal peptides from the library. Both components were dissolved in physiological phosphate buffer solution (1×PBS) with a pH value of 7.4 in a molar ratio of 1:4.5 (star-PEG:peptide) with a total concentration of 80 mg/ml. The reaction mixture was quickly covered and stirred at 750 rpm at room temperature for 18 hours (MR Hei-Standard, Heidolph, Schwabach, Deutschland) The raw products were analyzed through reverse-phase high pressure liquid chromatography (UPLC) (UPLC Aquity™ with UV detector, Waters, Milford, Mass., USA) by using C18 column (AQUITY™ UPLC BEH C18, grain size 1.7 μm, 50×2.1 mM, Waters, Milford, Mass., USA) and an isocratic gradient. The raw product was dialyzed with a dialysis membrane with cut-off limit (cut-off) of 8 kDa for two days against 10 liters of water under constant water exchange to release unbound peptides and salt. Thereafter, the product was again injected into the UPLC in order to examine the purity as compared to the analysis before the dialysis. The dialyzed product was dry frozen in water into a solid.

FIG. 3 shows the results of the reverse-phase high-pressure liquid chromatography (UPLC)-analysis of purified peptide four-arm-polyethyleneglycol-conjugate (peptide-star-PEG) by means of UV detection at 280 nm. The results are shown in FIG. 3 for the sample conjugates from the library a) KA7-star-PEG and in b) KG-star-PEG.

Following is the description of the production of the hydrogel networks. Hereby 14-kDa-heparin (25 mM, 2.5 mM) and peptide-star-PEG conjugates (6.25 mM, 3.125 mM) were dissolved in physiologic phosphate buffer solution (1×PBS) water or cell culture medium with 2% fetal bovine serum (FBS). These solutions were dissolved in a ratio of 1:4 heparin:peptide-star-PEG-conjugate by obtaining 0.5 mM or 5 mM 14-kDa-heparin and 2.5 mM or 5 mM peptide-star-PEG. The ligand/mol ratio was 2:1, 1:1 and 1:5 relative to the mol ratio of 14-kDa-heparin and the peptide-star-PEG-conjugate. The mixtures were incubated within a time frame of one hour to overnight at room temperature of 37° C. The gelling time spanned from present up to several hours depending on the applied peptide motif. A hydrogel was formed when it survived the addition of physiological phosphate buffer solution (1×PBS) pH 7.4 to the mixture after the incubation of the mixture over the prior night without mixing with the added solution.

Table 2 shows the selected peptides from the library, which reflect best the structural activity relationship of the hydrogel formation with heparin. ATIII is a heparin-binding peptide known from the literature. All peptides are connected to a four-armed, maleimide functionalized 10-kDa-polyethylenenglycol (Star-PEG). The hydrogel formation was tested in a 50 μl-mixture, which contains 5 mM 14-kDa-heparin and 5 mM (2.5 mM) star-PEG-peptide conjugate in physiological phosphate buffer solution (1×PBS) pH 7.4. The deformation and penetration speed was analyzed through centrifuging the hydrogel in a 45° table centrifuge with 275 μm metal balls at the surface. The deformation of the surface and the penetration of the metal balls were watched in dependence on the applied force.

TABLE 2

| Gel created | | Molecular | Peptide | | Pene- |
|---|---|---|---|---|---|
| with heparin | | weight | amount | Deformation | tration |
| | Peptide | [10⁻³ | [10⁻³ | speed | speed |
| Name | sequence | kg/mol] | mol/l] | [m/s²] | [m/s²] |
| ATIII | CWGGKAFAK | 2010,44 | 5 | nicht | nicht |
| | LAARLYRKA | | | bestimmt, Gel | bestimmt |
| | | | | schrurnpft | |
| KA5 | CWGGKAKAK | 1417,72 | 5 | 11223 +/− | 21209 +/− |
| | AKAKA | | | 4768 | 2188 |
| KA7 | CWGGKAKAK | 1816,22 | 2,5 | 43998 +/− | 72780 +/− |
| | AKAKAKAKA | | | 3139 | 6926 |
| KA7 | CWGGKAKAK | 1816,22 | 5 | >148317 | 138919 +/− |
| | AKAKAKAKA | | | | 16275 |
| RA5 | CWGGRARAR | 1557,82 | 5 | <687 | <687 |
| | ARARA | | | | |
| RA7 | CWGGRARAR | 2012,36 | 5 | 1069 +/− | 1952 +/− |
| | ARARARARA | | | 579 | 491 |

The heparin-binding domain of antithrombin III (ATIII) and heparin with low molecular weight can form a soft hydrogel if both are conjugated at star-PEG as described in N. Yamaguchi, B.-S. Chae, L. Zhang, K L Kiick, E M Furst, Biomacromolecules 2005, 6, 1931-1940. In order to reduce the chemical complexity, the investigations were carried out with 14 kDa-heparin. It was found that in the presence of ATM peptide which is conjugated to star-PEG (ATIII star-PEG) the resulting hydrogel is formed immediately but does not cover the total volume as shown in Table 2. The investigation of ATIII star-PEG and heparin showed that a strong interaction between heparin and peptide does not necessarily lead to an optimal hydrogel-network formation. Therefore, the library of peptide-star-PEG-conjugates was installed in order to investigate the peptide sequences of the heparin dependent self-organizing properties.

The (BA)ₙ sequence opens the possibility to change the peptide length and thus to slightly change the properties, which is the reason why this sequence was selected as a basis. As is known from R. Tyler-Cross, R B Harris, M. Sobel, D. Marques, Protein Science 1914 3, 620-627, the ATIII-peptide, after a heparin binding changes from a random coil to an α-helix wherein this is also expected for the (BA)ₙ and that it preferably transitions into an α-helix structure. It was surprisingly found that the (BA)ₙ motif shows a minimal sequence requirement for the interaction with heparin.

To follow the goal of attaining the greatest possible flexibility regarding properties, various repeats of (BA)ₙ were synthesized, that are set forth in the Tables 1 and 2. Single repeats were used as (negative) counter control because spiral-shaped formations require at least five amino acids (α-helix according to Pauling-Corey-Branson). To be able to compare also charge density dependencies besides length-charge-dependencies while recognizing binding to heparin, (BBA)₅ was synthesized (Table 1). (BBA)₅, at similar length exhibits a higher charge density than (BA)₇. As already stated, B and A have the tendency to form α-helical structures, as is known from C. Nick Pace, J. Martin Scholtz, Biophysical Journal 1998, 75, 422-427. To obtain always a tandem of potential structure forming and non-structure forming peptides, as Table 1 shows, each (BA)ₙ and (BBA)₅ had a (BG)ₙ- and a (BBG)₅-partner, wherein the letter G stands for Glycine. Glycine is known for interrupting any kind of structure formation. Thus, each of the intelligently configured members of the library had to fulfill a task.

In addition to the peptide motif, a tryptophan was labeled with a one letter code W, for UV detection and purification and a cysteine, marked with the one letter code C, bound to the N-terminal end of the peptide with two Glycines. By applying the Michael-addition-chemistry, the cysteine was coupled to the maleimide-functionalized 10-kDa-star-PEG. Synthesis and coupling of the peptide star-PEG-conjugates were optimized regarding purity, speed and simple handling as FIG. 3 shows. This is the largest library of peptide-polymer-conjugates, for which each of the oligosaccharide dependent hydrogel formation was analyzed. To analyze the formation of hydrogels, all peptide-star-PEG-conjugates were each mixed with 14-kDa-heparin in 50 μl physiological phosphate buffer (1×PBS) to an end concentration of 5 mM. After incubation overnight the physiological phosphate buffer (1×PBS) was added in order to analyze which mixtures formed a hydrogel. KA7-, KA5-, RA7- and RA5-star-PEG-conjugates with heparin did not mix with 1×PBS but formed a stable, clear hydrogel as Table 2 shows. These are the shortest de novo produced peptides known in the literature which from heparin dependent hydrogel.

In Table 3 peptides from the peptide library are shown that do not form a heparin dependent hydrogel. All peptides are coupled to a 10-kDa-maleimide-star-PEG. The gel formation was tested in a 50 μl-mixture which contains 5 mM 14 kDa-heparin and 5 mM peptide-star-PEG-conjugate in 1×PBS at a pH value of 7.4.

TABLE 3

| not created with heparin | | Molecular |
|---|---|---|
| Name | Peptide sequence | weight |
| KA1 | CWGGKA | 620,72 |
| KA3 | CWGGKAKAKA | 1019,22 |
| dKA7 | CWGGkAkAkAkAkAkAkA | 1816,22 |
| KdA7 | CWGGKaKaKaKaKaKaKa | 1816,22 |
| KA7-1a | CWGGKAKAKAKaKAKAKA | 1816,22 |
| KKA5 | CWGGKKAKKAKKAKKAKKA | 2058,57 |
| KG1 | CWGGKG | 606,69 |
| KG3 | CWGGKGKGKG | 977,13 |
| KG5 | CWGGKGKGKGKGKG | 1347,57 |
| KG7 | CWGGKGKGKGKGKGKGKG | 1718,01 |
| KKG5 | CWGGKKGKKGKKGKKGKKG | 1988,42 |
| RA1 | CWGGRA | 648,74 |
| RA3 | CWGGRARARA | 1103,28 |
| RRA5 | CWGGRRARRARRARRARRA | 2338,77 |
| RG1 | CWGGRG | 634,71 |
| RG3 | CWGGRGRGRG | 1061,19 |
| RG5 | CWGGRGRGRGRGRG | 1487,67 |
| RG7 | CWGGRGRGRGRGRGRGRG | 1914,15 |
| RRG5 | CWGGRRGRRGRRGRRGRRG | 2268,62 |

It is remarkable, that the (BBA)₅ forms no hydrogel with heparin although they exhibit a higher charge density as is shown in Table 3. This behavior must be based on the structure which were analyzed with the pure peptides.

The de novo produced heparin-binding peptides could be analyzed through application of circular dichroism spectroscopy (CD) (J-810, REV. 1.00, Jasco Inc. Eaton, Md., USA). All CD spectra were taken at wave lengths from 185 to 260 nm in a quartz cuvette of 1 mm optical path length. The data points were recorded at each nanometer in a response time of 4.0 s. All values of molar ellipticity [θ] are shown relative to the median number of peptide bonds in deg cm² dmol. FIGS. 4a to 4f contain the result of the analysis of heparin dependent structural change through circular dichroism spectroscopy (CD). Hereby the peptides were measured in MilliQ-water alone and together with 14 kDa heparin in a mol ratio of 1:1. Only for RA7 and KA7 twice as much heparin than peptide was used. The graph for the peptides that were mixed with 14-kDa-heparin, were corrected with the CD spectra of pure 14-kDa-heparin at the same concentration. The range for the peptide concentration was at 74.5 μM to 137.6 μM.

In Milli-Q-water (Advantage A10; Millipore GmbH) not only do (BG)ₙ- and (BBG)₅ motifs show a random coil structure, but also (BA)ₙ- and (BBA)₅ motifs as shown in FIGS. 4a to 4f. During observation of hydrogel formation of peptide-star-PEG-conjugate with heparin, the (BA)₇ motif and the (BA)₅ motif together with the heparin have shown a structural change. Due to the glycines, the (BG)ₙ motif and (BBG)₅ motif cannot change the structure in significant ways which is also expressed in the lack of hydrogel formation. RRA7 is the only peptide which while showing a structural change in the circular dichroism spectroscopy (CD), however, underwent no formation of hydrogel. Due to the denser charge distribution, the optimal distance of the positive charge of the peptide is not given and thus an optimal interaction with the sulfate of the heparin is not given. The (BA)ₙ peptide motif is thus preferred for heparin-binding peptides, although the (BBA)₅ peptide motif exhibits more positive charge at similar peptide length. This structure/activity relationship between the distance of the basic amino acids to alanine and the heparin binding capacity in the formation of hydrogels is in any event novel and surprising.

In order to underline the significance of the structure activity relationship of (BA)ₙ motifs, the mutants dKA7 and KdA7 were synthesized which are also set forth in Table 3. By mixing L- and D-amino acids the structure formation was supposed to the hindered. The analysis showed that these mutants show neither formation of a hydrogel coupled to a star-PEG and mixed with 14-kDa heparin (see Table 3) nor does one of these mutants show a structural change similar as the KA7 exhibits, which can also be gleaned from a comparison between FIGS. 4d and 4e. Likewise, the mutant KA7-1a having an D-alanine in exchange for the L-alanine, in the center of the peptide motif, forms no hydrogel, which is coupled with the star-PEG and is mixed with heparin, as also shown in Table 3. Also no structural change occurs in the circular dichroism (CD) as shown by FIG. 4e.

A comparative high-throughput analysis was performed regarding the mechanical properties of the hydrogels. In order to provide a fast high-throughput method for comparing small amounts of hydrogel, a tabletop centrifuge (5424R, Eppendorf, Hamburg, Germany) was used. For this purpose, 50 μl of the hydrogel were formed by mixing the peptide-star-PEG-conjugates and 14-kDa-heparin in physiological phosphate buffer (1×PBS, pH 7.4) to a final concentration of 5 mM (once 2.5 mM for KA7-star-PEG). The mixture was incubated in 0.2 ml reaction vessels over night. The deformation of the hydrogel surface was determined in reference to the 45° centrifuge rotor and the penetration of 275 μm metal spheres in dependence on the force that has to be produced by the centrifuge. All experiments were repeated three times.

FIG. 5 schematically shows the high-throughput analysis regarding the mechanical properties of the hydrogels. Hereby a) and b) show a deformation of the surface of the 50 μl hydrogel in a 0.2 ml reaction tube, more specifically a) below the speed required for deforming the surface, and b) at the speed required for deforming the surface. Part c) of FIG. 5 is a schematic representation of the penetration of a small spheres through 50 μl hydrogel in a 0.2 ml reaction tube depending on the force exerted by the 45° centrifuge.

It was shown that the RA5-PEG-hydrogel with heparin produced similar results as the mixtures with RRA7-star-PEG, which had not formed a hydrogel, see Tables 2 and 3. The RA7-, KA5- and KA7-based hydrogels are much stronger and according to Table 2 exhibit a broad range of stiffness. Arginine and lysine have different charge distributions on the side chain, which leads to different properties. The two different concentrations of KA7-star-PEG with heparin resulted in hydrogels with different mechanical properties as can be seen in Table 2. This shows that the hydrogel, which is based on a non-covalent peptide-biomolecule-interaction, can be adjusted in different ways. It is possible to experiment with the concentration of the components and with the peptide sequence. Mixtures of different (BA)ₙ-peptide-motifs on a star-PEG-molecule or different peptide-star-PEG-conjugates would even further improve the capability for adjustment in smaller steps.

FIG. 6 shows the stability of a lysine and arginine based hydrogel in comparison to L- and D-amino acids. The hydrogels were formed by mixing of the end concentrations of 5 mM peptide-star-PEG-conjugate and 5 mM 14-kDa-heparin in 50 μl physiological phosphate buffer (1×PBS). The analysis was performed by centrifuging the hydrogels in a 45° tabletop centrifuge with 275 μm metal spheres on the gel surface. The deformation of the surface and the penetration of the spheres were recorded in dependence on the exerted force.

An important result was that the complete change of the KA7 to D-amino acids has no influence on the hydrogel stiffness, as shown in FIG. 6. Due to the resistance of D-amino acids against proteases it is possible to create non-covalent hydrogels that are very stable in biological environments. In this way the degradability can be adjusted by different amino acids.

In order to test the broad spectrum of the mechanical properties according to Table 2, a rhelogical test was performed, which means the flow behavior of the KA7-star-PEG- and KA5-star-PEG-hydrogels with heparin was determined via frequency sweep and load sampling experiments.

FIG. 7a shows the amplitude course of the pure peptide-star-PEG-conjugate and the pure 14-kDa-heparin with a frequency of 1 Hz. FIG. 7b shows the frequency course of the pure peptide-star-PEG-conjugate and the pure 14-kDa-heparin with 2% amplitude. FIGS. 7c and 7d show the flow behavior of peptide-star-PEG-conjugate as mixture with 14-kDa-heparin. The final mixture in physiological phosphate buffer solution (1×PBS) contains of both 5 mM or 2.5 mM peptide-star-PEG-conjugate and 5 mM or 0.5 mM heparin. The solutions or the mixtures were analyzed by using a shear-stress controlled rheometer (MCR 301, Paar Physica, Anton Paar, Ashland, Va.) at 20° C. and a measuring unit with 39.979 mM diameter, an angle of 0.305° and a truncation of 24 μm.

The individual-component solutions of 14-kDa-heparin, KA5-star-PEG and KA7-star-PEG were analyzed in order to show the basic mechanical properties of the starting components in comparison to the mechanical properties of the mixtures. The individual-component-peptide-star-PEG-solutions were treated identically to the mixtures containing 14-kDa-heparin. All mixtures and solutions were incubated in an environment that was completely closed around the measuring unit to prevent evaporation. All incubation times were determined by gelling time experiments described below. The final mixture of 5 mM KA7-star-PEG and 5 mM 14-kDa-heparin was incubated for 1.5 hours. The final mixture of 2.5 mM KA7-star-PEG and 5 mM 14-kDa-heparin was incubated for 3 hours. The final mixture of 5 mM KA5-star-PEG and 5 mM 14-kDa-heparin was incubated for 15 hours. The amplitude course measurements were performed with a frequency of 1 Hz over a range from 0.1 to 100%. The frequency dependencies were detected by using a 1% amplitude and in a range of 0.01 to 100%. All experiments where repeated twice and the mean value plotted.

The storage modulus G′ was significantly higher for all samples than the loss modulus G″ (˜2%). These visco-elastic properties confirm that the interaction between the (KA)ₙ and heparin is very strong and stable. The stiffness of the pure 14 kDa-heparin or the pure peptide-star-conjugate is very low as shown in FIGS. 7a and 7b. The broad concentration spectrum of heparin that can be used ranges from 0.5 to 5 mM. Also the mechanical properties can be adjusted by a factor of more than 10 solely by changing the concentration of the components. The mixing of different peptide-star-PEG-conjugates is an additional way to change the gel properties. This provides two dimensions, the concentration and the peptide sequence that can be changed individually or together in order to adjust the hydrogel properties to the application at hand. This is possible solely based on the interaction of the peptide-motif (BA)ₙ with the biomolecule heparin.

In order to test the strength of the interaction between the peptide-motif and the 14 kDa-heparin the formed hydrogels were tested with regard to different solvents. Table 4 shows the result of this test of stability against different solvents. For this the peptide-star-PEG-conjugates were mixed with 14 kDa-heparin in 50 μl in physiological phosphate buffer solution (1×PBS) to a final concentration of respectively 5 mM. Each solvent, i.e., physiological phosphate buffer solution (1×PBS), Milli-Q-water, 1 M hydrochloric acid (HCl) 1 M sodium hydroxide solution (NaOH), saturated sodium chloride solution (NaCl), dimethyl sulfoxide (DMSO), ethanol and cell culture medium with 2% fetal bovine serum (FBS) were respectively added as 200 μl supernatant to the hydrogel. The hydrogel was incubated at a room temperature of 24° C. and the supernatants where exchanged every day for at least three days. All experiments were performed three times. Under none of the tested conditions the KA7-hydrogel could be destroyed. No other known none-covalent heparin-dependent hydrogel possesses such a stability, which emphasizes the extraordinarily stable interaction between the KA7 and heparin. Even the hydrogel on the basis of very short KA5 was only destroyed by 1 M HCl after more than one week incubation. 2,2,2-trifuourethanlol (TFE) is known to destroy any type of secondary structures. Even though KA7-star-PEG-hydrogel with heparin appears indestructible, the structure can be destroyed by adding 2,2,2-trifuourethanol (TFE) to the supernatant. Freeze-drying of this 2,2,2-trifluourethanol (TFE)-, KA7-star-PEG-, heparin-solution and the addition of physiological phosphate buffer solution (1×PBS) resulted in a clear gel again.

TABLE 4

| Supernatant | KA7 | KA5 | RA7 |
|---|---|---|---|
| 1 × PBS | Stable | Stable | Stable; the surface |
| | | | of the hydrogel |
| | | | was milky |
| Water | Stable | Stable | Stable |
| 1M HCl | Stable | Stable; hydrogel | Stable; hydrogel |
| | | was destroyed | was milky and |
| | | only after one | thereafter clear |
| | | week | again |
| 1M NaOH | Stable | Stable | Stable |
| Saturated NaCl | Stable | Stable | Not stable; |
| solution | | | hydrogel became |
| | | | milky |
| DMSO | Stable | Stable | Stable |
| Ethanol | Stable | Stable | Stable |
| Cell culture | stable | stable | stable |
| medium | | | |

For the hydrogel-formation stability test three respective different cases were tested under which the hydrogels are normally formed. Table 5 shows the test for forming the hydrogels in different solvents. The hydrogels were formed by mixing the final concentrations of respectively 5 mM peptide-star-PEG-conjugate and 5 mM 14-kDa-heparin in 50 μl physiological phosphate buffer solution (1×PBS), Milli-Q-water or cell culture medium with 2% fetal bovine serum (FBS). The stability of the hydrogels was tested with the same solvent in which it was formed in 200 μl supernatant. The hydrogel was incubated at room temperature (24° C.), the supernatants were changed every day for at least three days in a row and the result after at least 3 days analyzed.

TABLE 5

| Peptide-star-PEG- | | | cell culture |
|---|---|---|---|
| conjugate | PBS | water | medium |
| KA7 | formed | formed | formed |
| KA5 | formed | Formed | formed |
| RA7 | formed | formed | formed |

Cell culture medium with 2% fetal bovine serum (FBS) contains an amount of proteins and other components, which may potentially disrupt the interaction between the (BA)n-peptide-motif and 14-kDa-heparin if this interaction is not stable enough.

Further an erosion experiment was conducted. For this purpose peptide-star-PEG-conjugates were respectively mixed with TAMRA-marked 14-kDas-heparin. For the erosion experiment the TAMRA-marked 14-kDa heparin had to be synthesized beforehand. Hereby 14 kDa-heparin was marked with 5-(and-6)-carboxytetramethylrhodamin (TAMRA, Invitrogen) by using the 1-ethyl-3-(3-dimethylaminopropyl)carbodiimide/N-hydroxysulfosuccinimide-(EDAC/sNHS)-chemistry. Heparin, TAMRA, EDAC, sNHS and Na₂CO₃ were mixed at a ratio of 1:2:5:4:20 in water and incubated overnight. Thereafter the mixture was dialyzed for two days against 10 liters of water in a dialysis membrane with an exclusion limit of 8 kDa with constant exchange of water. The dialyzed product was filtered through a 0.22 μm polyvinylidene fluoride filter (PVDF-filter) and freeze-dried to a red product.

Subsequently the peptide-star-PEG-conjugates were respectively mixed with the TAMRA-marked 14-kDa-heaprin in 50 μl cell culture medium to a final concentration of 5 mM. The hydrogels were formed at 37° C. at 95% humidity and 5% CO₂ overnight (15 hours) (Galaxy 170S, Eppendorf, Hamburg Germany). After an incubation overnight 1 ml of the cell culture medium with 2% fetal bovine serum (FBS) was added. 200 μl of the supernatant was removed at each measuring time point and replaced with new cell culture medium with 2% fetal bovine serum (FBS). The fluorescence was measured at defined time points in the supernatant by using a plate reading device (BECKMAN COULTER PARADIGM Detection platform, BECKMAN COULTER, Brea, Calif., USA) and black 96-well-plates with clear bottom.

FIG. 8a shows the analysis of a heparin-dependent structural change by circular dichroism spectroscopy. Both peptides shoed without heparin a random coiled structure in Milli-Q-water. After addition of 14-kDa-heparin in a 2 molar concentration of the peptides a clear structural change occurred. FIG. 8b shows the result of the testing of the erosion of the hydrogel by the above-mentioned admixture of peptide-star-PEG-conjugate and TAMRA-marked 14-kD-heparin in 50 μl cell culture medium with 2% fetal bovine serum (FBS) to a final concentration of 5 mM. The fluorescence was measured in 200 μl of 1 ml supernatant. These 200 μl were each replaced by 200 μl fresh medium.

As is known from the literature, inter alia from J R Fromm, R E Hileman, E B O Caldwell, J M Weiler, R J Linhardt, Archives of Biochemistry and Biophysics 1997, 343, 92-100, arginine binds to heparin stronger than lysine. RA7 is bound stronger to heparin so that less heparin is released from the hydrogel with RA7-star-PEG than from the hydrogel with KA7-star-PEG. KA5 possesses less charge than KA7, so that the bond is weaker, which leads to more erosion. The hydrogels lost mass to a negligible degree so that it is likely that most of the heparin, which was released, i.e., up to 35%, is not part of the hydrogel network. After the stabilization of the heparin-erosion out of the hydrogel the latter is more stable than protein-hydrogels. The fact that the hydrogel remains very stable against serum and its components shows the specificity of the interaction between the (BA)ₙ-peptide-motif and heparin. Thus the hydrogel does not have to be preformed prior to application. This is a very significant advantage because it safes time and the concentration for example of the proteins is evenly distributed. In addition the reproducibility is greater because less production steps are involved.

For a scanning electron microscopic image, KA7-star-PEG-conjugates were mixed with 14-kDa-heparin to a final concentration of 5 mM respectively in 50 μl physiological phosphate buffer solution (1×PBS) and incubated at room temperature for three days. The sample was taken by inserting a capillary tube into the gel, shock-freezing in liquid nitrogen and cutting though the sample with a very sharp knife. The surface-dried and cut sample was imaged with a scanning electron microscope (Supra 40VP, Zeiss, Jena, Germany).

FIG. 9 shows a scanning electron microscopic image of the KA7-star-PEG-hydrogel with heparin. The sample was flash frozen in liquid nitrogen and analyzed after a short period of evaporation. The KA7-star-PEG-hydrogel showed a clear network structure.

In connection with the hydrogel preparation the gelling time is important. At the beginning the gelling time was to be determined by using the shear stress controlled rheometer (MCR 301, Paar Physica, Anton Paar, Ashland, Va.) at 20° C. and a measuring unit with 39,979 mm diameter, an angle of 0.305° and a truncation of 24 μm. Disadvantageously the measuring with 2% amplitude and a frequency of 1 Hz changed the gelling time. The gelling occurred much faster than was previously observed in the laboratory. This behavior necessitated a different approach to measure the gelling time. A microchip-controlled machine capable to measure the time dependent hydrogel stiffness on a fine scale (XP 205 Feingewicht Delta Range, Mettler-Toledo GmbH Giessen, Germany) was constructed and programmed. FIG. 10 shows a device for analyzing the gelling time of a hydrogel controlled by a programmable microchip and the use of a precision scale for measuring the gelling time of the hydrogels. Shown is a movable part on the precision scale consisting of a blunt needle and a holder for a 0.2 ml reaction vessel which contains the hydrogel mixture. A LabX-software, which was installed on a computer, was used to monitor and record the force.

Different concentrations of peptide-star-PEG-conjugates were mixed at constant stirring with 14-kDa-heparin, to form 50 μl hydrogel in physiological phosphate buffer solution (1×PBS) in the 0.2 ml reaction vessel. After the mixing the reaction vessel was closed with a lid having a 1.5 mm hole and the measuring started immediately. On the inside of the lid 10 μl of water protected the hydrogel surface from drying out. At the beginning of the measurement the blunt needle of 1 mm diameter is inserted 1 mm deep into the hydrogel viewed from above. Every 5 minutes the blunt needle moves 1 mm into the gel and after one second waiting time is moved upwards 30 μm below the original position. This 30 μm height difference ensures that the needle does not form a channel in the gel, which would not pose any resistance, but rather each measurement advances deeper and deeper into the gel (straight ahead from above) to always encounter an untouched hydrogel mixture, which can be measured. All data of the precision scale where monitored and documented by using the LabX software (Mettler-Toledo GmbH, Giessen, Germany) which was installed on a notebook and is connected with the precision scale with an RS-232 serial connection. The amplitudes of the resistance of the hydrogel against the pressure after pushing down the needle, corrected by the baseline prior to recording a measuring point, were plotted.

FIGS. 11a to 11c show the result of the analysis of the gelling time of the hydrogel by measuring the force required for inserting a needle with 1 mm diameter. Immediately after the mixing of the components the measurement was performed every 5 minutes. For each measurement the needle was moved downwards in the mixture by 1 mm and upwards by 0.970 mm. The force was measured by weighing and the amplitude, corrected by the baseline, was plotted.

Due to the different charge properties of RA7-star-PEG, KA7-star-PEG and KA5-star-PEG the gelling time differs. RA7-star-PEG-hydrogel forms with 14-kDa-heparin in physiological phosphate buffer (1×PBS) immediately with a final concentration of 5 mM. KA7-star-PEG requires about one hour for the formation of the hydrogel under the same conditions and KA5-star-PEG several hours. By lowering the concentration of the components, the gelling time increases as a comparison of FIGS. 11a and 11b shows. The mixing of different (BA)ₙ-peptide motifs which are coupled to star-PEG would make it possible to adjust stiffness and gelling time together. This provides a system for the user with which the gelling time can be adjusted by changing the concentration of the components or the ratio of the different star-PEG coupled (BA)ₙ-peptide motifs while retaining the solids content.

The hydrogel consisting of the (BA)ₙ-star-PEG-conjugates with heparin is not toxic to mammalian cells as could be shown in an in vitro cytotoxicity test of the hydrogel components (see FIGS. 12a to 12f. Because fibroblasts are the most important component of connective tissue, they were successfully used for a 9-day 3D cell culture by using KA7-star-PEG or KA-star-PEG with heparin in cell culture medium with 2% fetal bovine serum (FBS).

A frozen vial with cells was thawed in a 37° C. warm water bath for 2 minutes. The cells were always transferred into 5 ml complete cell culture medium 106 (with 2% fetal bovine serum (FBS)). This cell suspension was centrifuged at 700 g in a centrifuge (ROTINA 380 R, Hettich, Tuttlingen, Germany), the supernatant removed and the cells resuspended in 6 ml complete cell culture medium. After the mixing the suspension was transferred into a cell culture container and incubated at 37° C., 95% humidity and 5% CO₂. After 2 days the medium was changed until the cells were confluent. The cell culture medium of the confluent phase of the cells was removed and 1 ml trypsin/EDTA solution was added to the cell layer. After 5 minutes and occasional shaking the cells are in suspension and 3 ml of the new culture medium was added. This 4 ml cell suspension was diluted in 20 ml of the complete cell culture medium and transferred into 4 new cell culture containers (each 6 ml). Thereafter they were further incubated as described, until use or further division.

The cell culture medium from the confluent layer of the cells was removed and 1 ml of the trypsin/EDTA solution was added to the cell layer. After 5 minutes and occasional shaking the cells are in suspension and 3 ml of the new complete cell culture medium was added. The amount of the cells was counted by mixing 50 μl of the cell suspension with 50 μl of the trypan blue solution, wherein the number of cells was obtained with a hemocytometer. The 4 ml of the cell suspension were centrifuged at 700 g in a centrifuge (ROTINA 380 R, Hettich, Tuttlingen, Germany) the supernatant removed and the cells suspended in an amount of complete cell culture medium to obtain the target concentration. The tests for toxicity of the peptide-star-PEG-conjugates were performed by inoculating 5000 HDFn cells per well in a 96 well plate. After the transfer of the cells they were able to attach to the 96 well plates. The cells were incubated 24 hours prior to administering the samples at 37° C., 5% CO₂ and 95% humidity.

FIGS. 12a to 12f show a toxicity test for different peptide-star-PEG-conjugates and 14-kDa-heaprin. The cell medium was replaced after the 24 hours incubation by 200 μl of a solution containing fresh medium and 10⁻⁴ or 10⁻⁵ M peptide-star-PEG-conjugate, which was filtered through 0.22 μm centrifuge tube filters. Thus 10⁴ and 10-⁵ M peptide-star-PEG-conjugate or heparin was added to the 5000 resuspended human fibroblasts in cell culture medium with serum. After the addition of the entire test samples the cells were incubated for a further 24 hours at 37° C., 5% CO₂ and 95% humidity in order to analyze the time-dependent and also the concentration-dependent cytotoxicity. At the end of each exposure the toxicity level of each test sample was evaluated by a test with 3-(4,5 dimethylazol-2-yl)-2,5-diphenyltetrazoliumbromide (MTT) in order to determine the cytotoxicity of the peptide-star-PEG-conjugates compared to non-treated cells. Hereby the yellow 3-(4,5-dimethylthiazol-2-yl)-2,5-diphenyltetrazoliumbromide (MTT) is reduced to violet-colored formazan. In the same way all steps were performed without the addition of the compound to the cells. Thus the cells, which were incubated with medium alone, were used as control.

The MTT test aids in judging the viability of the cells by measuring the enzymatic reduction of yellow tetrazolium to violet formazan crystals. After incubation of the cells with the test sample MTT was added and incubated for a further 4 hours. After 4 hours the medium was removed and 100 μl of Dimethylsulfoxide (DMSO) was added in order to dissolve the formazan crystals, which were produced from the reduction of the tetrazolium salt solely by the metabolically active cells. The absorption of the solubilized formazan crystals was measured at 570 nm by using a plate reader (BECKMAN COULTER PARADIGM Detection platform, BECKMAN COULTER, Brea, Calif., USA). Because the absorption directly indicates the number of viable cells, the percentage of viability was calculated directly from the absorption values. The average toxicity was calculated by the mean value of 15 wells of the cells, which were treated with the same compound.

FIGS. 12a to 12f show the results of the MTT tests for a) 14-kDa-heparin b) ATIII-star-PEG c-conjugate, c) KA5-star-PEG-conjugate, d) RA5-star-PEG-conjugate, e) KA7-star-PEG-conjugate and f) RA7-star-PEG-conjugate.

For embedding the cells in the hydrogel the cell medium was first removed from the confluent layer of the cells and then 1 ml trypsin/EDTA solution added to the cell layer. After 5 minutes and occasional shaking the cells are in suspension, whereupon 3 ml of new cell culture medium were added. The amount of cells was counted by mixing of 50 μl of the cell suspension with 50 μl of trypan blue solution, wherein the cells were counted by using a hemocytometer. The 4 ml of cell suspension were centrifuged at 700 g in a centrifuge (ROTINA 380 R, Hettich, Tuttlingen, Germany) the supernatant removed and the cells resuspended in an amount of complete cell culture medium to obtain the target concentration. KA7-star-PEG-conjugates were dissolved in the entire cell culture medium and filtered through a 0.22 μm centrifuge tube filter. The same was performed with 14-kDa-heparin. To the solution of KA7-star-PEG-conjugate cells were correspondingly added to obtain a final concentration of 10⁶ cells per milliliter. Thereafter the KA7-star-PEG-conjugat-cell-mixture was mixed with 14 kDa-heparin to a final concentration of 5 mM of both in 50 μl, pipetted onto the bottom of an 8-well-plate and incubated overnight at 37° C., 95% humidity and 5% CO₂. After 1 day 0.5 ml of the entire cell culture medium was added (and changed every 2 days) and the cells were further incubated at 37° C., 95% humidity and 5% CO₂. The embedding of the cells in the KA5-star-PEG-conjugate-mixture with 14-kDa-heaprin was performed in the same manner as in the case of the KA7-star-PEG-conjugate with similar results.

The viability of the cells was determined by addition of 50 μl MTT 3-(4,5-dimethylazol-2-yl)-2,5-diphenyltetrazoliumbromide) into the 500 μl of the complete cell culture medium, as supernatant of the cells embedded in the hydrogel. The cells were imaged after 1 hour of incubation with a dissection microscope.

The viability of the cells was examined with a so-called Live/Dead® Assay. The cells-containing gels were rinsed twice with physiological phosphate buffer solution (1×PBS). A solution of 10 μM probidiumidodine (PI) (Molecular probes, Invitrogen, Germany) and 0.15 μM fluorescine diacetate (FDA) (Fluka, Germany) in physiological phosphate buffer solution (1×PBS) were applied for three minutes onto the gels, followed by rinsing with 1×PBS. The cells were imaged with a confocal microscope (Leica SP5, 10×/04). The images were recorded for a gel section of 100 μm thickness and the maximum intensity projection (MIP) of the images shown.

Prior to the immuno-staining the samples were fixed with 4% paraformaldehyde (PFA) for 15 minutes at room temperature and blocked in 0.25% bovine serum albumin (BSA) (Sigma-Aldrich, Munich, Germany) and 1% Triton-X100 (Sigma-Aldrich, Munich Germany) in PBS). Next Phalloidin-CF488 (Biotrend, Germany) was applied in blocking buffer for 5 minutes. Thereafter 0.1 μg/ml 4′,6diamidino-2-phenylindol (DAPI, Sigma-Aldrich, Munich, Germany) was applied in 1×PBS for 5 minutes, followed by 3×15 minutes of washing with buffer. The samples were imaged with a confocal microscope (Leica SP5, 63×/1.4-0.6).

FIG. 13 shows in a) to f) the results of the viability test and the structure of the human fibroblasts (HDFn) from the skin of newborns in a hydrogel. The final mixture in complete cell culture medium contains 5 mM KA5-star-PEG-conjugate and 14-kDa-heparin for the parts a) to c) and 2.5 mM KA7-star-PEG-conjugate and 14-kDa-heparin for the parts d) and f) of FIG. 13. The concentration of the cells was 10⁶ cells per ml. the parts a) and d) of FIG. 13 show wide-field microscopic images, scale 1 mM, of HDFn stained with 3-(4,5-dimethylthiazol-2-yl)-2,5-diphenyltetrazoliumbromide) (MTT) in the hydrogel. The parts b) and e) of FIG. 13 show Live/Dead® Assay stained HDFn. Part b) of FIG. 13 shows a proportion of live cells of 99 −+1%. The parts c) and f) of FIG. 13 show actin filaments and the nucleus of HDFn stained with pholloidin-CF488 and DAPI. The parts b) and c) and also the parts e) and f) of FIG. 13 show microscopic confocal laser scanning images.

In summary, the hydrogel matrix according to the invention enables biological functions, as well as non-toxicity for human cells, protein binding and adjustable enzymatic degradability together with flexible physical properties, such as adjustability of the gelling time and flow behavior by variation of the oligosaccharides and peptides (peptide-star-PEG-conjugates) and their concentration and broad chemical modifiability purely by non-covalent interaction of the hydrogel matrix components without any chemical reaction during the gel formation.

In particular heparin is a highly sulfated glycosaminoglycane, which binds growth factors, which are for example used in cell culture. Non-covalent hydrogels that are based on heparin and paring binding peptides were developed, however they are not adjustable. De novo produced heparin-binding peptides whose properties can be changed by adjusting their length, solve this problem. All properly functioning peptides that have been produced de novo so far are longer than 20 amino acids. This would lead to synthesis problems when additional properties are to be introduced. Here, the variety of the properties of the newly configured (BA)ₙ-peptide-motif was demonstrated by using a designed peptide-star-PEG-library for non-covalent hydrogel formation. Not only do these peptides undergo structural changes after binding to heparin as reported for peptide of natural origin, but they also represent the shortest artificial heparin binding peptides that are known from the literature. The adjustability of the non-covalent hydrogel with (BA)ₙ-star-PEG-conjugates and heparin is surprising. It is possible to adjust the stiffness, the gelling time and the biological and chemical stability solely by changing the length, the concentration or the type of basic amino acid of the (BA)ₙ peptide-motif. In this way it is possible to change the properties while retaining the solids content or to change the solids content while keeping the properties stable.

1. A non-covalent self-organizing hydrogel matrix for biotechnological applications, containing a covalent polymer-peptide-conjugate, wherein the covalent polymer-peptide-conjugate comprises conjugates of two or more peptides which are coupled to a polymer chain, and the peptide-sequence contains a repetitive dipeptide-motif (BA)ₙ, in which B is an amino acid with a positive side chain, A is alanine and n is a number between 4 and 20.

2. The non-covalent self-organizing hydrogel matrix according to claim 1, wherein the polymer chain is formed by a linear or multi arm polyethylene glycol (PEG).

3. The non-covalent self-organizing hydrogel matrix according to claim 2, wherein the polymer chain is formed by a four arm polyethylene glycol (star-PEG).

4. The non-covalent self-organizing hydrogel matrix according to one of the claims 1-3, wherein it further comprises a highly negatively charges oligosaccharide and the hydrogel matrix in configured in the form of an oligosaccharide/peptide/[polymer-system.

5. The non-covalent self-organizing hydrogel matrix according to claim 4, wherein the highly negatively charged oligosaccharide is a sulfated or phosphorylated oligosaccharide.

6. The non-covalent self-organizing hydrogel matrix according to claim 5, wherein the highly negatively charged oligosaccharide is selected from the group of oligosaccharides including heparin, dextransulfate, α-cyclodextrin sulfate, β-cyclodextrinphosphate, γ-cyclodextrin sulfate, α-cyclodextrin phosphate, β-cyclodextrin phosphate and γ-cyclodextrin phosphate.

7. The non-covalent self-organizing hydrogel matrix according to claim 6, wherein the heparin originates from mucosal tissue of pig intestine or bovine lung.

8. The non-covalent self-organizing hydrogel matrix according to claim 6, wherein dextransulfate has a molecular weight in the range of 4 kDa to 600 kDa.

9. The non-covalent self-organizing hydrogel matrix according to claim 6, wherein the degree of sulfation in the α-cyclodextrin sulfate, β-cyclodestrine sulfate or γ-cyclodextrine sulfate is from three sulfates per molecule to the complete sulfation.

10. The non-covalent self-organizing hydrogel matrix according to claim 6, wherein the degree of phosphorylation in the α-cyclodextrin sulfate, β-cyclodestrine sulfate or γ-cyclodextrine sulfate is from three phosphate groups per molecule to the complete phosphorylation.

11. The non-covalent self-organizing hydrogel matrix according to claim 1, further comprising a chemical group cleaved by light and situated between the polymer chain and the peptide sequence, and which contains the repetitive dipeptide motif (BA)ₙ.

12. The non-covalent self-organizing hydrogel matrix, further comprising a pH-sensitive chemical linker between the polymer chain and the peptide sequence, which contains the repetitive dipeptide motif (BA)ₙ.

13. The non-covalent self-organizing hydrogel matrix according to claim 1, further comprising an enzymatically cleavable linker between the polymer chain and the peptide chain which contains the repetitive dipeptide motif (BA)ₙ.

14. The non-covalent self-organizing hydrogel matrix according to claim 13, further comprising as enzymatically cleavable linker a peptide sequence which is a proteolytically active substrate.

15. The non-covalent self-organizing hydrogel matrix according to claim 13, further comprising as enzymatically cleavable linker an oligonucleotide sequence which is a nuclease-active substrate.

16. The non-covalent self-organizing hydrogel matrix according to claim 1 having an elasticity modulus of at least 10 Pa.

17. Hydrogel beads, formed from a non-covalent hydrogel matrix according to claim 16.

18. A combination of a non-covalent self-organizing hydrogel matrix according to claim 16 with cells embedded in the hydrogel matrix.

19. The combination according to claim 18, wherein the cells are selected from the group consisting of mammalian cells, insect cells, bacterial cells and yeast cells.

20. The combination according to claim 19, wherein the cells are mammalian cells, selected from the group of different cancer cell lines, fibroblast cells, pluripotent stem cells, induced pluripotent stem cells, human T-cells and human B-cells.

21. A method of using the combination according to claim 20 for the production of proteins, said proteins including therapeutic monoclonal antibodies.

22. A capsule for targeted release of therapeutic agents, comprising the non-covalent self-organizing hydrogel matrix according to claim 16.

23. The capsule according to claim 22, wherein agents are selected from a group including mammalian cells, insect cells, bacteria, yeast cells, anti-cancer-compounds, anti-coagulation compounds, inflammation inhibiting compounds, immunosuppressive compounds, therapeutic antibodies, diagnostic agents, hormones, growth factors, small molecules as inhibitor for cytokines, aptamer-inhibitors for growth factors and aptamer-inhibitors for cytokines.

24. The composition of a non-covalent self-organizing hydrogel matrix according to claim 16 and chemicals and therapeutic agents, wherein a gradient of the chemicals and agents is generated in the therapeutic hydrogel.

25. The composition according to claim 24 wherein the chemicals and therapeutic agents, which form gradients in the non covalent self organizing hydrogel matrix, are selected from a group including anti-coagulation compounds, inflammation inhibiting compounds, immunosuppressive compounds, therapeutic antibodies, diagnostic agents, hormones, growth factors, small molecules as inhibitors for cytokines, aptamer-inhibitors for growth factors and aptamer-inhibitors for cytokines.

26. A hybrid system from a non-spherical non-covalent self-organizing hydrogel matrix according to claim 1, wherein the hydrogel matrix and the hydrogel beads each have a different chemical composition and a component of the hybrid system is adjustable by irradiation with light, by selective chemical degradation or by enzymatic digestion.

27. A combination of hydrogel beads according to claim 17 with cells embedded in the hydrogel beads.
